SEQUENCE LISTING (45 sequences)

SEQ ID NO: 1 - protein, 19 aa, Artificial Sequence
  Synthetic Construct
MSRLPVLLLLQLLVRPGLQ

SEQ ID NO: 2 - protein, 21 aa, Artificial Sequence
  Synthetic Construct
MALPVTALLLPLALLLHAARP

SEQ ID NO: 3 - protein, 45 aa, Artificial Sequence
  Synthetic Construct
TTTPAPRPPTPAPTIASQPLSLRPEACRPAAGGAVHTRGLDFACD

SEQ ID NO: 4 - protein, 24 aa, Artificial Sequence
  Synthetic Construct
IYIWAPLAGTCGVLLLSLVITLYC

SEQ ID NO: 5 - protein, 42 aa, Artificial Sequence
  Synthetic Construct
KRGRKKLLYIFKQPFMRPVQTTQEEDGCSCRFPEEEEGGCEL

SEQ ID NO: 6 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 128 aa, Artificial Sequence
  Synthetic Construct
VTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLPI

SEQ ID NO: 8 - protein, 128 aa, Artificial Sequence
  Synthetic Construct
IPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 9 - protein, 133 aa, Artificial Sequence
  Synthetic Construct
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 10 - protein, 25 aa, Artificial Sequence
  Synthetic Construct
KEELEAEKRDLIRTNERLSQELEYL

SEQ ID NO: 11 - protein, 21 aa, Artificial Sequence
  Synthetic Construct
GSGEGRGSLLTCGDVEENPGP

SEQ ID NO: 12 - protein, 23 aa, Artificial Sequence
  Synthetic Construct
SGGGGSGGGGSGGGGSGSGGGGS

SEQ ID NO: 13 - protein, 22 aa, Artificial Sequence
  Synthetic Construct
SGGGGSGSGGGGSGGGGSGGGG

SEQ ID NO: 14 - protein, 22 aa, Artificial Sequence
  Synthetic Construct
GGGGSGGGGSGGGGSGSGGGGS

SEQ ID NO: 15 - protein, 24 aa, Artificial Sequence
  Synthetic Construct
GGGGSGGGGSGGGGGGSGSGGGGS

SEQ ID NO: 16 - protein, 704 aa, Artificial Sequence
  Synthetic Construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTSGGGGSGGGGSGGGGSGSGGGGSRRVTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLPISGGGGSGGGGSGGGGSGSGGGGSIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDDSGGGGSGSGGGGSGGGGSGGGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 700 aa, Artificial Sequence
  Synthetic Construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTSGGGGSGGGGSGGGGSGSGGGGSVTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLPISGGGGSGGGGSGGGGSGSGGGGSIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSGGGGSGSGGGGSGGGGSGGGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 552 aa, Artificial Sequence
  Synthetic Construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTSGGGGSGGGGSGGGGGGSGSGGGGSKEELEAEKRDLIRTNERLSQELEYLVTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 527 aa, Artificial Sequence
  Synthetic Construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTSGGGGSGGGGSGGGGGGSGSGGGGSVTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 554 aa, Artificial Sequence
  Synthetic Construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTSGGGGSGGGGSGGGGGGSGSGGGGSKEELEAEKRDLIRTNERLSQELEYLIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 2112 bp, Artificial Sequence
  Synthetic Construct
atgagtagactgcccgtgctgctgctgctgcagctgctggtgcgccccggactgcaggccccaatgacccagacaacacctctgaaaacctcttgggtgaactgcagcaatatgatcgacgagatcatcacacacctgaagcagccccctctgccactgctggatttcaacaatctgaacggcgaggaccaggatatcctgatggagaacaatctgagacggcccaacctggaggcctttaatagggccgtgaagagcctgcagaacgccagcgccatcgagtccatcctgaagaatctgctgccatgtctgcctctggcaaccgcagcaccaacacgccacccaatccacatcaaggacggcgattggaacgagttcaggcgcaagctgaccttttacctgaagacactggagaatgcccaggcccagcagaccacactgtccctggccatcttcacctccggcggcggcggctctggaggaggaggaagcggaggaggaggatctggatctggcggaggaggctctcggagagtgacacggcagatgtgcatctataccaaccccacaagctgtaatgagatctacggcaagtttagctccgcctatctggcctgcgacggcaagcagatggagatcatcaccctgctgaacccttctctgatcagcggcgatgagtggcagtggtccggcaatacaccaatccacgtgctgggcatgtggcactactctaaggtgctgaagctgctggaccaggatgagaagtcctatgtgaagctgctgtctgccaaccagtccatgtgctctgcccagagcgagtactggaataagagcgaggacttctttcagtactgcaacaataagtattgtatctggaacaagccatgccccggctgttattccgagacactgcctatctctggcggaggaggatccggcggcggcggctccggcggcggaggaagcggctccggcggcggcggcagcatcccactgacagagtcctactgcggcccttgtccaaagaattggatctgctacaagaacaactgttaccagttctttgatgagagcaagaactggtatgagtcccaggcctcttgcatgagccagaatgcctctctgctgaaggtgtacagcaaggaggaccaggatctgctgaagctggtgaagagctatcactggatgggcctggtgcacatccccaccaacggctcctggcagtgggaggacggctccatcctgtctcctaatctgctgacaatcatcgagatgcagaagggcgattgtgccctgtacgcctctagcttcaagggctatatcgagaactgctccacccctaatacatacatctgtatgcagcggaccgtggacgattctggcgggggaggcagtgggtcagggggagggggaagcggaggaggagggagcggcggggggggcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - DNA, 2094 bp, Artificial Sequence
  Synthetic Construct
atgagccgactgcccgtgctgctgctgctgcagctgctggtgcgacccggactgcaggcccccatgacccagactaccccactgaaaacctcttgggtgaactgcagcaatatgatcgacgagatcatcacacacctgaagcagccccctctgccactgctggatttcaacaatctgaacggcgaggaccaggatatcctgatggagaacaatctgagacggcccaacctggaggcctttaatcgggccgtgaagagcctgcagaacgccagcgccatcgagtccatcctgaagaatctgctgccatgtctgcctctggcaaccgcagcaccaacaagacacccaatccacatcaaggacggcgattggaacgagttcaggcgcaagctgaccttttacctgaagacactggagaatgcccaggcccagcagaccacactgtccctggccatcttcacctccggcggcggcggctctggaggaggaggaagcggaggaggaggatctggatctggcggaggaggctccgtgacaaggcagatgtgcatctataccaaccccacatcttgtaatgagatctacggcaagtttagctccgcctatctggcctgcgacggcaagcagatggagatcatcaccctgctgaacccttctctgatcagcggcgatgagtggcagtggagcggcaatacaccaatccacgtgctgggcatgtggcactactccaaggtgctgaagctgctggaccaggatgagaagtcctatgtgaagctgctgtctgccaaccagtccatgtgctctgcccagagcgagtactggaataagtccgaggacttctttcagtactgcaacaataagtattgtatctggaacaagccatgccccggctgttattctgagacactgcctatctctggcggaggaggatccggcggcggcggctccggcggcggaggaagcggctccggcggcggcggcagcatcccactgacagagtcctactgcggcccttgtccaaagaattggatctgctacaagaacaactgttaccagttctttgatgagagcaagaactggtatgagtcccaggcctcttgcatgagccagaatgcctctctgctgaaggtgtacagcaaggaggaccaggatctgctgaagctggtgaagtcttatcactggatgggcctggtgcacatccccaccaacggcagctggcagtgggaggacggctccatcctgtctcctaatctgctgacaatcatcgagatgcagaagggcgattgtgccctgtacgcctctagcttcaagggctatatcgagaactgctccacccctaatacatacatctgtatgcagagaaccgtgtctgggggagggggaagcggaagtggcggaggcggctctggcgggggaggaagtggagggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 23 - DNA, 1656 bp, Artificial Sequence
  Synthetic Construct
atgagtagactgcccgtgctgctgctgctgcagctgctggtgcgccccggactgcaggccccaatgacccagacaaccccactgaagacctcttgggtgaactgcagcaatatgatcgacgagatcatcacacacctgaagcagccccctctgccactgctggatttcaacaatctgaacggcgaggaccaggatatcctgatggagaacaatctgagacggcccaacctggaggcctttaatcgcgccgtgaagtctctgcagaacgccagcgccatcgagtccatcctgaagaatctgctgccatgtctgccactggcaaccgcagcacctacacggcacccaatccacatcaaggacggcgattggaacgagttcaggcgcaagctgaccttttacctgaagacactggagaatgcccaggcccagcagaccacactgagcctggccatcttcacctccggcggcggcggctctggaggaggaggaagcggcggaggaggaggaggctctggcagcggcggcggcggctctaaggaggagctggaggccgagaagcgggacctgatcagaaccaatgagaggctgagccaggagctggagtacctggtgacacggcagatgtgcatctataccaaccctacatcctgtaatgagatctacggcaagtttagctccgcctatctggcctgcgacggcaagcagatggagatcatcaccctgctgaacccctccctgatctctggcgatgagtggcagtggagcggcaatacacctatccacgtgctgggcatgtggcactactccaaggtgctgaagctgctggaccaggatgagaagtcctatgtgaagctgctgtctgccaaccagagcatgtgctccgcccagtctgagtactggaataagtccgaggatttctttcagtattgtaacaacaaatactgcatctggaacaaaccctgtcccggctgctactcagagaccctgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 24 - DNA, 1581 bp, Artificial Sequence
  Synthetic Construct
atgagtagactgcccgtgctgctgctgctgcagctgctggtgcgccccggactgcaggccccaatgacccagacaaccccactgaaaacctcttgggtgaactgcagcaatatgatcgacgagatcatcacacacctgaagcagccccctctgccactgctggatttcaacaatctgaacggcgaggaccaggatatcctgatggagaacaatctgagacggcccaacctggaggcctttaatcgggccgtgaagtccctgcagaacgccagcgccatcgagtccatcctgaagaatctgctgccatgtctgccactggcaaccgcagcacctacaaggcacccaatccacatcaaggacggcgattggaacgagttcaggcgcaagctgaccttttacctgaagacactggagaatgcccaggcccagcagaccacactgtctctggccatcttcacctccggcggcggcggctctggaggaggaggaagcggcggaggaggaggaggctctggcagcggcggcggcggcagcgtgacacggcagatgtgcatctataccaaccctacatcctgtaatgagatctacggcaagtttagctccgcctatctggcctgcgacggcaagcagatggagatcatcaccctgctgaacccctccctgatctctggcgatgagtggcagtggtctggcaatacacctatccacgtgctgggcatgtggcactacagcaaggtgctgaagctgctggaccaggatgagaagtcctatgtgaagctgctgtctgccaaccagagcatgtgctccgcccagtctgagtactggaataagagcgaggacttctttcagtattgtaacaacaagtattgcatttggaacaagccctgccccgggtgctattctgaaacactgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 25 - DNA, 1662 bp, Artificial Sequence
  Synthetic Construct
atgagtagactgcccgtgctgctgctgctgcagctgctggtgcgacccggcctgcaggctccaatgacccagacaaccccactgaagacctcttgggtgaactgcagcaatatgatcgacgagatcatcacacacctgaagcagccccctctgcctctgctggatttcaacaatctgaacggcgaggaccaggatatcctgatggagaacaatctgagacggcccaacctggaggcctttaatcgcgccgtgaagagcctgcagaacgccagcgccatcgagtccatcctgaagaatctgctgccttgtctgccactggcaaccgcagcaccaacacggcaccctatccacatcaaggacggcgattggaacgagttcaggcgcaagctgaccttttacctgaagacactggagaatgcccaggcccagcagaccacactgtccctggccatcttcacctccggcggcggcggctctggaggaggaggaagcggcggaggaggaggaggctctggcagcggcggcggcggctctaaggaggagctggaggccgagaagcgggacctgatcagaaccaacgagaggctgagccaggagctggagtacctgatccccctgacagagtcctattgcggcccatgtcccaagaattggatctgctacaagaacaactgttaccagttctttgatgagtccaagaactggtacgagtcccaggcctcttgcatgagccagaatgcctccctgctgaaggtgtactctaaggaggaccaggatctgctgaagctggtgaagtcttatcactggatgggcctggtgcacatcccaaccaacggcagctggcagtgggaggacggctccatcctgtctcccaatctgctgacaatcatcgagatgcagaagggcgattgtgccctgtatgccagctccttcaaagggtatatcgagaattgctccactccaaacacttacatctgtatgcagcggaccgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - DNA, 2295 bp, Artificial Sequence
  Synthetic Construct
atggctctgcccgtgaccgccctgctgctgcccctggctctgctgctgcacgccgcccgccctgtgacaagacagatgtgcatctataccaaccccacatcctgcaatgagatctacggcaagttcagctccgcctatctggcctgtgacggcaagcagatggagatcatcaccctgctgaacccatctctgatcagcggcgatgagtggcagtggtccggcaatacacccatccacgtgctgggcatgtggcactactctaaggtgctgaagctgctggaccaggatgagaagtcttatgtgaagctgctgagcgccaaccagtccatgtgctctgcccagagcgagtactggaataagtccgaggacttctttcagtactgcaacaataagtattgtatctggaacaagccatgccccggctgttattctgagacactgcccatcagcggaggaggaggatccggcggaggaggctctggcggcggcggctccggctctggaggaggaggatccatccctctgacagagtcttactgcggcccttgtccaaagaattggatctgctacaagaacaactgttaccagttctttgatgagtccaagaactggtatgagtcccaggcctcttgtatgagccagaatgccagcctgctgaaggtgtactccaaggaggaccaggatctgctgaagctggtgaagagctatcactggatgggcctggtgcacatccctaccaacggctcctggcagtgggaggacggctccatcctgtctccaaatctgctgacaatcatcgagatgcagaagggcgattgcgccctgtacgcctctagcttcaagggctatatcgagaactgcagcaccccaaatacatacatctgtatgcagcgcaccgtgaccacaaccccagcacctcggccccctaccccagcaccaacaatcgcaagccagcctctgtccctgcgcccagaggcatgtaggccagcagcaggaggagcagtgcacaccagaggcctggactttgcctgcgatatctatatctgggcacctctggcaggaacctgtggcgtgctgctgctgagcctggtcatcaccctgtactgcaagagaggcaggaagaagctgctgtatatcttcaagcagccctttatgcgccctgtgcagacaacccaggaggaggacggctgctcctgtaggttcccagaagaggaggagggaggatgtgagctgagagtgaagtttagcaggtccgccgatgcacctgcataccagcagggacagaaccagctgtataacgagctgaatctgggccggagagaggagtacgacgtgctggataagaggaggggacgggaccccgagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccaccaagaggatctggagagggaaggggcagcctgctgacatgcggcgacgtggaggagaaccctggcccaatgagcagactgccagtgctgctgctgctgcagctgctggtgaggcccggcctgcaggcacctatgacccagacaacccccctgaagacaagctgggtgaactgttccaatatgatcgacgagatcatcacccacctgaagcagcctccactgcctctgctggatttcaacaatctgaatggcgaggaccaggatatcctgatggagaacaatctgagaaggccaaacctggaggcctttaatagagccgtgaagtctctgcagaacgcctctgccatcgagagcatcctgaagaatctgctgccttgcctgccactggcaaccgcagcaccaacaaggcaccccatccacatcaaggacggcgattggaacgagttccgccggaagctgaccttttacctgaagacactggagaatgcccaggcccagcagacaaccctgagcctggccatcttcacaaccacaccagcacctcgccccccaactcctgccccaacaatcgcatcccagccactgtctctgcgccccgaggcatgcaggcctgcagcaggcggcgccgtgcacacccggggcctggactttgcctgtgatatctacatctgggcccccctggccggaacttgtggcgtcctgctgctgtccctggtcatcactctgtattgc

SEQ ID NO: 27 - DNA, 1818 bp, Artificial Sequence
  Synthetic Construct
atggctctgcccgtgactgccctgctgctgcctctggctctgctgctgcatgctgctcgcccaatccctctgactgaatcatactgtggcccatgccccaagaactggatctgctacaagaacaattgttatcagttctttgacgagtctaagaactggtacgagtcccaggcctcttgtatgagccagaatgcctctctgctgaaggtgtacagcaaggaggaccaggatctgctgaagctggtgaagagctatcactggatgggcctggtgcacatccccaccaacggctcctggcagtgggaggacggctccatcctgtctcctaatctgctgacaatcatcgagatgcagaagggcgattgcgccctgtacgccagctccttcaagggctatatcgagaactgcagcaccccaaatacatacatctgtatgcagaggaccgtgaccacaacccctgcaccacggccccctaccccagcacctacaatcgcaagccagccactgtccctgcgccccgaggcatgtaggcctgcagcaggcggcgccgtgcacaccagaggcctggactttgcctgcgatatctatatctgggcacctctggcaggaacctgtggcgtgctgctgctgagcctggtcatcaccctgtactgcaagagaggcaggaagaagctgctgtatatcttcaagcagccttttatgcgcccagtgcagacaacccaggaggaggacggctgctcttgtcggttcccagaggaggaggagggcggctgtgagctgagagtgaagttttctaggagcgccgatgcaccagcataccagcagggacagaaccagctgtataacgagctgaatctgggccggagagaggagtacgacgtgctggataagaggaggggacgggaccccgagatgggaggcaagccacggagaaagaacccccaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccaccaagaggatccggagagggcaggggctctctgctgacatgcggcgatgtggaggagaacccaggccccatgtccagactgcctgtgctgctgctgctgcagctgctggtgaggcctggcctgcaggcaccaatgacccagacaaccccactgaagacaagctgggtgaactgttccaatatgatcgacgagatcatcacccacctgaagcagcctccactgcccctgctggatttcaacaatctgaatggcgaggaccaggatatcctgatggagaacaatctgagaaggcctaacctggaggcctttaatagagccgtgaagagcctgcagaacgcctctgccatcgagagcatcctgaagaatctgctgccatgcctgccactggcaaccgcagcacccacaaggcaccctatccacatcaaggacggcgattggaacgagttccgccggaagctgaccttttatctgaagacactggagaatgcccaggcccagcagacaaccctgtccctggccatcttcacaaccacacctgcaccacgccccccaactcctgcccctacaatcgcatcccagccactgtctctgcgccctgaggcatgtcggccagccgccggaggagccgtgcacacccggggcctggatttcgcttgtgacatctacatttgggctcctctggctggcacctgtggggtcctgctgctg

SEQ ID NO: 28 - protein, 1 aa, Artificial Sequence
  Synthetic Construct
  VARIANT (1)...(1): can be present in repeat of at least 1
G

SEQ ID NO: 29 - protein, 2 aa, Artificial Sequence
  Synthetic Construct
  VARIANT (1)...(2): can be present in repeat of at least 1
GS

SEQ ID NO: 30 - protein, 5 aa, Artificial Sequence
  Synthetic Construct
  VARIANT (1)...(5): can be present in repeat of at least 1
GSGGS

SEQ ID NO: 31 - protein, 4 aa, Artificial Sequence
  Synthetic Construct
  VARIANT (1)...(4): can be present in repeat of at least 1
GGGS

SEQ ID NO: 32 - protein, 5 aa, Artificial Sequence
  Synthetic Construct
  VARIANT (1)...(5): can be present in repeat of at least 1
GGGGS

SEQ ID NO: 33 - protein, 516 aa, Artificial Sequence
  Synthetic Construct
MALPVTALLLPLALLLHAARPIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGSGGGSGGGSGGGSIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 523 aa, Artificial Sequence
  Synthetic Construct
MALPVTALLLPLALLLHAARPVTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLPISGGGGSGGGGSGGGGSGSGGGGSIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 372 aa, Artificial Sequence
  Synthetic Construct
MALPVTALLLPLALLLHAARPIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 765 aa, Artificial Sequence
  Synthetic Construct
MALPVTALLLPLALLLHAARPVTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLPISGGGGSGGGGSGGGGSGSGGGGSIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTTTPAPRPPTPAPTIASQPLSLRPEACRPAAGGAVHTRGLDFACDIYIWAPLAGTCGVLLLSLVITLYC

SEQ ID NO: 37 - protein, 606 aa, Artificial Sequence
  Synthetic Construct
MALPVTALLLPLALLLHAARPIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTTTPAPRPPTPAPTIASQPLSLRPEACRPAAGGAVHTRGLDFACDIYIWAPLAGTCGVLLL

SEQ ID NO: 38 - DNA, 1554 bp, Artificial Sequence
  Synthetic Construct
atggctctgcccgtgactgccctgctgctgcctctggctctgctgctgcatgctgctcgcccaatccctctgactgaatcatactgtggcccatgccccaagaactggatctgctacaagaacaattgttatcagttctttgacgagtctaagaactggtacgagtcccaggcctcttgtatgagccagaatgcctctctgctgaaggtgtacagcaaggaggaccaggatctgctgaagctggtgaagagctatcactggatgggcctggtgcacatccccaccaacggctcctggcagtgggaggacggctccatcctgtctcctaatctgctgacaatcatcgagatgcagaagggcgattgcgccctgtacgccagctccttcaagggctatatcgagaactgcagcaccccaaatacatacatctgtatgcagaggaccgtgggaggaggaagcggaggaggatccggaggcggctctggcggcggcagcatccctctgactgaatcatactgtggcccatgccccaagaactggatctgctacaagaacaattgttatcagttctttgacgagtctaagaactggtacgagtcccaggcctcttgtatgagccagaatgcctctctgctgaaggtgtacagcaaggaggaccaggatctgctgaagctggtgaagagctatcactggatgggcctggtgcacatccccaccaacggctcctggcagtgggaggacggctccatcctgtctcctaatctgctgacaatcatcgagatgcagaagggcgattgcgccctgtacgccagctccttcaagggctatatcgagaactgcagcaccccaaatacatacatctgtatgcagaggaccgtgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 39 - DNA, 1569 bp, Artificial Sequence
  Synthetic Construct
atggctctgcccgtgaccgccctgctgctgcccctggctctgctgctgcacgccgcccgccctgtgacaagacagatgtgcatctataccaaccccacatcctgcaatgagatctacggcaagttcagctccgcctatctggcctgtgacggcaagcagatggagatcatcaccctgctgaacccatctctgatcagcggcgatgagtggcagtggtccggcaatacacccatccacgtgctgggcatgtggcactactctaaggtgctgaagctgctggaccaggatgagaagtcttatgtgaagctgctgagcgccaaccagtccatgtgctctgcccagagcgagtactggaataagtccgaggacttctttcagtactgcaacaataagtattgtatctggaacaagccatgccccggctgttattctgagacactgcccatcagcggaggaggaggatccggcggaggaggctctggcggcggcggctccggctctggaggaggaggatccatccctctgacagagtcttactgcggcccttgtccaaagaattggatctgctacaagaacaactgttaccagttctttgatgagtccaagaactggtatgagtcccaggcctcttgtatgagccagaatgccagcctgctgaaggtgtactccaaggaggaccaggatctgctgaagctggtgaagagctatcactggatgggcctggtgcacatccctaccaacggctcctggcagtgggaggacggctccatcctgtctccaaatctgctgacaatcatcgagatgcagaagggcgattgcgccctgtacgcctctagcttcaagggctatatcgagaactgcagcaccccaaatacatacatctgtatgcagcgcaccgtgaccacaaccccagcacctcggccccctaccccagcaccaacaatcgcaagccagcctctgtccctgcgcccagaggcatgtaggccagcagcaggaggagcagtgcacaccagaggcctggactttgcctgcgatatctatatctgggcacctctggcaggaacctgtggcgtgctgctgctgagcctggtcatcaccctgtactgcaagagaggcaggaagaagctgctgtatatcttcaagcagccctttatgcgccctgtgcagacaacccaggaggaggacggctgctcctgtaggttcccagaagaggaggagggaggatgtgagctgagagtgaagtttagcaggtccgccgatgcacctgcataccagcagggacagaaccagctgtataacgagctgaatctgggccggagagaggagtacgacgtgctggataagaggaggggacgggaccccgagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacctatgatgccctgcacatgcaggccctgccaccaaga

SEQ ID NO: 40 - DNA, 1122 bp, Artificial Sequence
  Synthetic Construct
atggctctgcccgtgactgccctgctgctgcctctggctctgctgctgcatgctgctcgcccaatccctctgactgaatcatactgtggcccatgccccaagaactggatctgctacaagaacaattgttatcagttctttgacgagtctaagaactggtacgagtcccaggcctcttgtatgagccagaatgcctctctgctgaaggtgtacagcaaggaggaccaggatctgctgaagctggtgaagagctatcactggatgggcctggtgcacatccccaccaacggctcctggcagtgggaggacggctccatcctgtctcctaatctgctgacaatcatcgagatgcagaagggcgattgcgccctgtacgccagctccttcaagggctatatcgagaactgcagcaccccaaatacatacatctgtatgcagaggaccgtgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 41 - protein, 221 aa, Artificial Sequence
  Synthetic Construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTTTPAPRPPTPAPTIASQPLSLRPEACRPAAGGAVHTRGLDFACDIYIWAPLAGTCGVLLLSLVITLYC

SEQ ID NO: 42 - protein, 213 aa, Artificial Sequence
  Synthetic Construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFTTTPAPRPPTPAPTIASQPLSLRPEACRPAAGGAVHTRGLDFACDIYIWAPLAGTCGVLLL

SEQ ID NO: 43 - DNA, 663 bp, Artificial Sequence
  Synthetic Construct
atgagcagactgccagtgctgctgctgctgcagctgctggtgaggcccggcctgcaggcacctatgacccagacaacccccctgaagacaagctgggtgaactgttccaatatgatcgacgagatcatcacccacctgaagcagcctccactgcctctgctggatttcaacaatctgaatggcgaggaccaggatatcctgatggagaacaatctgagaaggccaaacctggaggcctttaatagagccgtgaagtctctgcagaacgcctctgccatcgagagcatcctgaagaatctgctgccttgcctgccactggcaaccgcagcaccaacaaggcaccccatccacatcaaggacggcgattggaacgagttccgccggaagctgaccttttacctgaagacactggagaatgcccaggcccagcagacaaccctgagcctggccatcttcacaaccacaccagcacctcgccccccaactcctgccccaacaatcgcatcccagccactgtctctgcgccccgaggcatgcaggcctgcagcaggcggcgccgtgcacacccggggcctggactttgcctgtgatatctacatctgggcccccctggccggaacttgtggcgtcctgctgctgtccctggtcatcactctgtattgc

SEQ ID NO: 44 - DNA, 639 bp, Artificial Sequence
  Synthetic Construct
atgtccagactgcctgtgctgctgctgctgcagctgctggtgaggcctggcctgcaggcaccaatgacccagacaaccccactgaagacaagctgggtgaactgttccaatatgatcgacgagatcatcacccacctgaagcagcctccactgcccctgctggatttcaacaatctgaatggcgaggaccaggatatcctgatggagaacaatctgagaaggcctaacctggaggcctttaatagagccgtgaagagcctgcagaacgcctctgccatcgagagcatcctgaagaatctgctgccatgcctgccactggcaaccgcagcacccacaaggcaccctatccacatcaaggacggcgattggaacgagttccgccggaagctgaccttttatctgaagacactggagaatgcccaggcccagcagacaaccctgtccctggccatcttcacaaccacacctgcaccacgccccccaactcctgcccctacaatcgcatcccagccactgtctctgcgccctgaggcatgtcggccagccgccggaggagccgtgcacacccggggcctggatttcgcttgtgacatctacatttgggctcctctggctggcacctgtggggtcctgctgctg

SEQ ID NO: 45 - protein, 16 aa, Artificial Sequence
  Synthetic Construct
IYIWAPLAGTCGVLLL

(19) United States
(12) Patent Application Publication
(10) Pub. No.: US 2021/0363218 A1
(43) Pub. Date: Nov. 25, 2021

(54) MULTISPECIFIC CHIMERIC RECEPTORS COMPRISING AN NKG2D DOMAIN AND METHODS OF USE THEREOF

(71) Applicant: Nanjing Legend Biotech Co. Ltd., Nanjing (CN)

(72) Inventors: Xiaohu FAN, Edmonton (CA); Jun WANG, Nanjing (CN); Pingyan WANG, Nanjing (CN); Qiuchuan ZHUANG, Nanjing (CN); Lian MA, Nanjing (CN)

(21) Appl. No.: 16/957,790

(22) PCT Filed: Dec. 28, 2018
(86) PCT No.: PCT/CN2018/124978

§ 371 Date: Jun. 25, 2020

(30) Foreign Application Priority Data
Dec. 28, 2017 (CN) PCT/CN2017/119397

(51) Int. Cl. C07K 14/705; C07K 16/24; A61K 35/17

(52) U.S. Cl. CPC C07K 14/7056; A61K 38/00; A61K 35/17; C07K 16/244

(57) ABSTRACT

Provided are chimeric receptors targeting NKG2D, and multispecific chimeric receptors comprising an NKG2D domain and a second antigen binding domain such as an IL-3 domain. Also provided are dual chimeric receptor systems comprising a first chimeric receptor comprising an NKG2D domain, and a second chimeric receptor comprising a second antigen binding domain such as an IL-3 domain. Further provided are engineered immune effector cells (such as T cells), pharmaceutical compositions, kits and methods of treating cancer.

### CROSS REFERENCE TO RELATED APPLICATIONS

This application claims priority benefit of International Patent Application No. PCT/CN2017/119397 filed Dec. 28, 2017, the contents of which are incorporated herein by reference in their entirety.

### SUBMISSION OF SEQUENCE LISTING ON ASCII TEXT FILE

The contents of the following submission on ASCII text file are incorporated herein by reference in their entirety: a computer readable form (CRF) of the Sequence Listing (file name: 761422000941SEQLISTING.txt, date recorded: Dec. 28, 2018, size: 85 KB).

### FIELD OF THE PRESENT APPLICATION

The present invention relates to chimeric receptors, multispecific chimeric receptors, dual chimeric receptor systems, engineered immune effector cells, and methods of use thereof.

### BACKGROUND OF THE PRESENT APPLICATION

Acute myeloid leukemia (AML) is characterized by aberrant clonal proliferation of myeloid precursors that dominate the bone marrow and blood, which severely impairs normal hematopoiesis. AML is the most common type of acute leukemia, accounting for roughly 25% of all adult-onset leukemia in the Western world. Occurring at an incidence of 3-5 per 100,000 adults per year, AML has the highest death rate among all leukemia (DiNardo and Cortes 2016). In the last 40 years, the five-year relative overall survival rate for AML patients has increased slightly from a dismal 6.3% between 1975 to 1980 to 23.9% between 2007 to 2012 (Mardiros et al. 2015).

The standard first line treatment for AML is chemotherapy using cytarabine in combination with an anthracycline as induction therapy, followed by repeated cycles of high-dose cytarabine and/or an allogenic stem cell transplant (alloSCT) for patients who achieve complete remission (CR) after the induction therapy. Although initial CR can be achieved by current induction chemotherapy in almost 70% of young adult patients, 43% of patients will eventually relapse, and 18% never attain CR using first line induction treatment (Forman and Rowe 2013). AlloSCT is the preferred treatment method following a second remission. The five-year disease-free survival rate among patients receiving alloSCT is 40-50%, which demonstrates susceptibility of AML to immune-based therapy. However, patients having primary refractory disease or having an initial CR lasting fewer than 6 months benefit little from alloSCT treatment (Mardiros et al. 2015). Additionally, cytotoxic damage to the organs of patients by conventional salvage chemotherapy further compromises the chance of success for alloSCT. Therefore, more effective and less toxic therapeutics are needed for AML patients after relapse or induction failure.

T cells have the potential to attack and eradicate tumors, especially those with high mutational burden that result in neo-antigens. However, the antitumor capability of T cells is often actively inhibited by an immune-suppressive tumor microenvironment (TME) (McGranahan et al. 2016). Chimeric antigen receptor T cells (CAR-T) are constructed by transducing genes encoding fusion proteins comprising an extracellular antigen binding domain directed at an antigen on tumor cells, a hinge region, and an intracellular signaling domain of T cell receptor (TCR) to induce T cell activation upon antigen binding. Different from conventional T cells, which rely on native TCRs for tumor antigen recognition, CAR-T cells are redirected to unprocessed antigens, thereby killing tumor cells independently from their expression of major histocompatibility complex (MHC) antigens. Therefore, CAR-T cells have the ability to overcome many inherent limitations of immunotherapy. After two decades of preclinical research and clinical trials, the safety and feasibility of CAR-T based therapy have been confirmed, and unprecedented clinical results have been obtained in hematological malignancies (Kochenderfer et al. 2015; Louis et al. 2011).

The disclosures of all publications, patents, patent applications and published patent applications referred to herein are hereby incorporated herein by reference in their entirety.

### BRIEF SUMMARY OF THE PRESENT APPLICATION

The present application provides multispecific chimeric receptors and dual chimeric receptor systems that target NKG2D ligands and a second antigen, such as a tumor antigen, e.g., CD123. Chimeric receptors targeting NKG2D ligands are also provided.

One aspect of the present application provides a chimeric receptor comprising: (a) an extracellular domain comprising an NKG2D domain; (b) a transmembrane domain; and (c) an intracellular signaling domain. In some embodiments, the extracellular domain comprises a first NKG2D domain and a second NKG2D domain.

One aspect of the present application provides a multispecific chimeric receptor comprising: (a) an extracellular domain comprising an NKG2D domain and a second antigen binding domain; (b) a transmembrane domain; and (c) an intracellular signaling domain.

One aspect of the present application provides a multispecific chimeric receptor comprising a polypeptide chain comprising: (a) an extracellular domain comprising a first NKG2D domain, a second NKG2D domain and a second antigen binding domain; (b) a transmembrane domain; and (c) an intracellular signaling domain. In some embodiments, the extracellular domain comprises from the N-terminus to the C-terminus: the second antigen binding domain, the first NKG2D domain and the second NKG2D domain. In some embodiments, the second antigen binding domain is fused to the first NKG2D domain via a peptide linker. In some embodiments, the peptide linker is no more than about 50 amino acids long. In some embodiments, the peptide linker comprises an amino acid sequence selected from SEQ ID NOs: 12-15.

One aspect of the present application provides a multispecific chimeric receptor comprising a first polypeptide chain and a second polypeptide chain each comprising: (a) an extracellular domain comprising an NKG2D domain and a second antigen binding domain; (b) a transmembrane domain; and (c) an intracellular signaling domain. In some embodiments, each extracellular domain further comprises a dimerization motif. In some embodiments, the dimerization motif is displaced between the NKG2D domain and the second antigen binding domain. In some embodiments, the dimerization motif is a leucine zipper or a cysteine zipper. In some embodiments, the second antigen binding domain is fused to the NKG2D domain via a peptide linker. In some embodiments, the peptide linker is no more than about 50 amino acids long. In some embodiments, the peptide linker comprises an amino acid sequence selected from SEQ ID NOs: 12-15. In some embodiments, the NKG2D domain of the first polypeptide chain is crosslinked to the NKG2D domain of the second polypeptide chain via one or more disulfide bonds. In some embodiments, each of the first polypeptide chain and the second polypeptide chain comprises from the N-terminus to the C terminus: the second antigen binding domain, the NKG2D domain, the transmembrane domain, and the intracellular signaling domain.

In some embodiments according to any one of the multispecific chimeric receptors described above, the multispecific chimeric receptor is a bispecific chimeric receptor.

In some embodiments according to any one of the multispecific chimeric receptors described above, the second antigen binding domain is an antibody fragment. In some embodiments, the antibody fragment specifically binds to an antigen selected from the group consisting of CD19, CD20, CD22, CD33, CD38, BCMA, CS1, ROR1, GPC3, CD123, CD138, c-Met, EGFR, EGFRvIII, HER2, HER3, GD-2, NY-ESO-1, MAGE A3, and glycolipid F77. In some embodiments, the second antigen binding domain is a ligand or a ligand binding domain. In some embodiments, the ligand or ligand binding domain is derived from a molecule selected from the group consisting of NKG2A, NKG2C, NKG2F, IL-3, IL-13, LLT1, AICL, DNAM-1, and NKp80. In some embodiments, the second antigen binding domain is an IL-3 domain. In some embodiments, the IL-3 domain comprises the amino acid sequence of SEQ ID NO: 9, or a variant thereof having at least about 85% (e.g., at least about 90%, 92%, 95%, 98%, or 99%) sequence identity to the amino acid sequence of SEQ ID NO: 9.

In some embodiments according to any one of the chimeric receptors or multispecific chimeric receptors described above, the NKG2D domain, or the first NKG2D domain and/or the second NKG2D domain comprises the amino acid sequence of SEQ ID NO: 7 or 8, or a variant thereof having at least about 85% (e.g., at least about 90%, 92%, 95%, 98%, or 99%) sequence identity to the amino acid sequence of SEQ ID NO: 7 or 8.

In some embodiments according to any one of the chimeric receptors or multispecific chimeric receptors described above, the transmembrane domain is derived from a molecule selected from the group consisting of CD8a, CD4, CD28, 4-1BB, CD80, CD86, CD152 and PD1. In some embodiments, the transmembrane domain comprises the amino acid sequence of SEQ ID NO: 4 or 45.

In some embodiments according to any one of the chimeric receptors or multispecific chimeric receptors described above, the intracellular signaling domain comprises a primary intracellular signaling domain of an immune effector cell. In some embodiments, the primary intracellular signaling domain is derived from CD3ζ. In some embodiments, the primary intracellular signaling domain comprises the amino acid sequence of SEQ ID NO: 6.

In some embodiments according to any one of the chimeric receptors or multispecific chimeric receptors described above, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, ICOS, CD30, CD40, CD3, LFA-1, CD2, CD7, LIGHT, NKG2C, B7-H3, Ligands of CD83 and combinations thereof. In some embodiments, the co-stimulatory signaling domain comprises a cytoplasmic domain of CD28 and/or a cytoplasmic domain of 4-1BB. In some embodiments, the co-stimulatory signaling domain comprises the amino acid sequence of SEQ ID NO: 5.

In some embodiments according to any one of the chimeric receptors or multispecific chimeric receptors described above, the chimeric receptor or multispecific chimeric receptor further comprises a hinge region located between the C-terminus of the extracellular domain and the N-terminus of the transmembrane domain. In some embodiments, the hinge region is derived from CD8a. In some embodiments, the hinge region comprises the amino acid sequence of SEQ ID NO: 3.

In some embodiments, there is provided one or more isolated nucleic acid(s) comprising a nucleic acid sequence encoding one or more of the polypeptide chains in any one of the chimeric receptors or multispecific chimeric receptors described above.

One aspect of the present application provides a dual chimeric receptor system comprising: (i) a first chimeric receptor comprising a first polypeptide chain and a second polypeptide chain each comprising: (a) a first extracellular domain comprising an NKG2D domain; (b) a first transmembrane domain; and (c) a first intracellular signaling domain; and (ii) a second chimeric receptor comprising a third polypeptide chain comprising: (a) a second extracellular domain comprising a second antigen binding domain; and (b) a second transmembrane domain. In some embodiments, the second chimeric receptor further comprises a second intracellular signaling domain.

One aspect of the present application provides a dual chimeric receptor system comprising: (i) a first chimeric receptor comprising a first polypeptide chain comprising: (a) a first extracellular domain comprising a first NKG2D domain and a second NKG2D domain; (b) a first transmembrane domain; and (c) a first intracellular signaling domain; and (ii) a second chimeric receptor comprising a second polypeptide chain comprising: (a) a second extracellular domain comprising a second antigen binding domain; and (b) a second transmembrane domain. In some embodiments, the second chimeric receptor further comprises a second intracellular signaling domain.

In some embodiments according to any one of the dual chimeric receptor systems described above, the second antigen binding domain is an antibody fragment. In some embodiments, the antibody fragment specifically binds to an antigen selected from the group consisting of CD19, CD20, CD22, CD33, CD38, BCMA, CS1, ROR1, GPC3, CD123, CD138, c-Met, EGFR, EGFRvIII, HER2, HER3, GD-2, NY-ESO-1, MAGE A3, and glycolipid F77. In some embodiments, the second antigen binding domain is a ligand or a ligand binding domain. In some embodiments, the ligand or ligand binding domain is derived from a molecule selected from the group consisting of NKG2A, NKG2C, NKG2F, IL-3, IL-13, LLT1, AICL, DNAM-1, and NKp80. In some embodiments, the second antigen binding domain is an IL-3 domain. In some embodiments, the IL-3 domain comprises the amino acid sequence of SEQ ID NO: 9, or a variant thereof having at least about 85% (e.g., at least about 90%, 92%, 95%, 98%, or 99%) sequence identity to the amino acid sequence of SEQ ID NO: 9.

In some embodiments according to any one of the dual chimeric receptor systems described above, the NKG2D domain, or the first NKG2D domain and/or the second NKG2D domain comprises the amino acid sequence of SEQ ID NO: 7 or 8, or a variant thereof having at least about 85% (e.g., at least about 90%, 92%, 95%, 98%, or 99%) sequence identity to the amino acid sequence of SEQ ID NO: 7 or 8.

In some embodiments according to any one of the dual chimeric receptor systems described above, the first and/or second transmembrane domain is derived from a molecule selected from the group consisting of CD8a, CD4, CD28, 4-1BB, CD80, CD86, CD152 and PD1. In some embodiments, the first and/or second transmembrane domain comprises the amino acid sequence of SEQ ID NO: 4 or 45.

In some embodiments according to any one of the dual chimeric receptor systems described above, the first and/or second intracellular signaling domain comprises a primary intracellular signaling domain of an immune effector cell. In some embodiments, the primary intracellular signaling domain is derived from CD3ζ. In some embodiments, the primary intracellular signaling domain comprises the amino acid sequence of SEQ ID NO: 6.

In some embodiments according to any one of the dual chimeric receptor systems described above, the first and/or second intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the co-stimulatory signaling domain comprises a cytoplasmic domain of CD28 and/or a cytoplasmic domain of 4-1BB. In some embodiments, the co-stimulatory signaling domain comprises the amino acid sequence of SEQ ID NO: 5.

In some embodiments according to any one of the dual chimeric receptor systems described above, the first and/or second chimeric receptor further comprises a hinge region located between the C-terminus of the extracellular domain and the N-terminus of the transmembrane domain. In some embodiments, the hinge region is derived from CD8a. In some embodiments, the hinge region comprises the amino acid sequence of SEQ ID NO: 3.

In some embodiments, there is provided one or more isolated nucleic acid(s) comprising a nucleic acid sequence encoding one or more polypeptide chains in any one of the dual chimeric receptor systems described above. In some embodiments, there is provided an isolated nucleic acid comprising a first nucleic acid sequence encoding the first chimeric receptor and a second nucleic acid sequence encoding the second chimeric receptor, wherein the first nucleic acid sequence is operably linked to the second nucleic acid sequence via a third nucleic acid sequence encoding a self-cleaving peptide. In some embodiments, the self-cleaving peptide is a T2A, P2A, or F2A peptide.

In some embodiments, there is provided a chimeric receptor comprising an amino acid sequence selected from the group consisting of SEQ ID NOs: 16-20 and SEQ ID NOs: 33-35, or a variant thereof having at least about 85% (e.g., at least about 90%, 92%, 95%, 98%, or 99%) sequence identity to the amino acid sequence selected from the group consisting of SEQ ID NOs: 16-20 and 33-35. In some embodiments, there is provided a dual chimeric receptor system comprising a first chimeric receptor comprising an amino acid sequence of SEQ ID NO: 34, or a variant thereof having at least about 85% (e.g., at least about 90%, 92%, 95%, 98%, or 99%) sequence identity to the amino acid sequence of SEQ ID NO: 34; and a second chimeric receptor comprising an amino acid sequence of SEQ ID NO: 41, or a variant thereof having at least about 85% (e.g., at least about 90%, 92%, 95%, 98%, or 99%) sequence identity to the amino acid sequence of SEQ ID NO: 41. In some embodiments, there is provided a dual chimeric receptor system comprising a first chimeric receptor comprising an amino acid sequence of SEQ ID NO: 35, or a variant thereof having at least about 85% (e.g., at least about 90%, 92%, 95%, 98%, or 99%) sequence identity to the amino acid sequence of SEQ ID NO: 35; and a second chimeric receptor comprising an amino acid sequence of SEQ ID NO: 42, or a variant thereof having at least about 85% (e.g., at least about 90%, 92%, 95%, 98%, or 99%) sequence identity to the amino acid sequence of SEQ ID NO: 42. In some embodiments, there is provided a polypeptide comprising an amino acid sequence of SEQ ID NO: 36 or 37, or a variant thereof having at least about 85% (e.g., at least about 90%, 92%, 95%, 98%, or 99%) sequence identity to the amino acid sequence of SEQ ID NO: 36 or 37. In some embodiments, there is provided an isolated nucleic acid comprising a nucleic acid sequence selected from the group consisting of SEQ ID NOs: 21-27 and 38-40, or a variant thereof having at least about 85% (e.g., at least about 90%, 92%, 95%, 98%, or 99%) sequence identity to the nucleic acid sequence selected from the group consisting of SEQ ID NOs: 21-27 and 38-40.

In some embodiments, there is provided one or more vectors encoding any one or more of the isolated nucleic acids described above. In some embodiment, the vector is a lentiviral vector.

Another aspect of the present application provides an engineered immune effector cell, comprising any one of the chimeric receptors, the multispecific chimeric receptors or the dual chimeric receptor systems, the isolated nucleic acids, or the vectors described above. In some embodiments, the immune effector cell is a T cell, an NK cell, a peripheral blood mononuclear cell (PBMC), a hematopoietic stem cell, a pluripotent stem cell, or an embryonic stem cell. In some embodiments, the immune effector cell is a T cell.

In some embodiments, there is provided a pharmaceutical composition, comprising any one of the engineered immune effector cells described above, and a pharmaceutically acceptable carrier.

Another aspect of the present application provides a method of treating cancer in an individual, comprising administering to the individual an effective amount of any one of the pharmaceutical compositions described above. In some embodiments, the cancer is multiple myeloma, acute lymphoblastic leukemia, or chronic lymphocytic leukemia.

Also provided are methods of use, kits, and articles of manufacture comprising any one of the chimeric receptors, multispecific chimeric receptors, dual chimeric receptor systems, engineered immune effector cells, isolated nucleic acids, or vectors described above.

### BRIEF DESCRIPTION OF THE DRAWINGS

FIG. 1A shows a schematic view of an exemplary bispecific chimeric receptor (LIC2001) comprising a single polypeptide chain comprising an IL-3 domain and two NKG2D domains. A positively charged residue (R) is engineered at the N terminus of the first NKG2D domain (e.g., a reverse NKG2D domain), and a negatively charged residue (D) is engineered at the C terminus of the second NKG2D domain (e.g., a forward NKG2D domain). The engineered R and D residues form a salt bridge with each other to promote dimerization.

FIG. 1B shows a schematic view of an exemplary bispecific chimeric receptor (LIC2001-1) comprising a single polypeptide chain comprising an IL-3 domain and two NKG2D domains.

FIG. 1C shows a schematic view of exemplary bispecific chimeric receptors comprising two polypeptide chains each comprising an IL-3 domain, a leucine zipper motif, and an NKG2D domain. The leucine zipper motif of each polypeptide chain promotes dimerization. Additionally, the NKG2D domains can be crosslinked to each other via disulfide bonds. LIC2002 comprises an IL-3 domain, a leucine zipper motif, and a reverse NKG2D domain. LIC2002-2 comprises an IL-3 domain, a leucine zipper motif, and a forward NKG2D domain.

FIG. 1D shows a schematic view of an exemplary bispecific chimeric receptor (LIC2002-1) comprising two polypeptide chains each comprising an IL-3 domain and an NKG2D domain. The NKG2D domains are crosslinked to each other via disulfide bonds.

FIG. 1E shows a schematic view of an exemplary dual chimeric receptor system (LIC2003) comprising a first chimeric receptor targeting NKG2D ligands, and a second chimeric receptor targeting CD123. The first chimeric receptor comprises a polypeptide chain comprising two NKG2D domains, which can be crosslinked to each other via disulfide bonds. The second chimeric receptor comprises an IL-3 domain. The second chimeric receptor may or may not contain an intracellular signaling domain.

FIG. 1F shows a schematic view of an exemplary dual chimeric receptor system (LIC2004) comprising a first chimeric receptor targeting NKG2D ligands, and a second chimeric receptor targeting CD123. The first chimeric receptor comprises two polypeptide chains each comprising a single NKG2D domain, wherein the NKG2D domains are crosslinked to each other via disulfide bonds. The second chimeric receptor comprises an IL-3 domain. The second chimeric receptor may or may not contain an intracellular signaling domain. This figure shows an exemplary second IL-3 chimeric receptor having no intracellular signaling domain.

FIG. 2 shows expression of NKG2D×IL-3 chimeric receptor constructs (LIC2004 and LIC2002-2) in engineered T cells as determined by flow cytometry.

FIGS. 3A-3C show in vitro cytotoxic activities of engineered T cells expressing various NKG2D×IL-3 chimeric receptor constructs against tumor cells: K562-CD123-Luc (FIG. 3A), K562-Luc (FIG. 3B) and KG1-Luc (FIG. 3C).

FIG. 4 shows a plot comparing dose-dependent cytotoxic activities of engineered T cells expressing various chimeric receptor constructs against K562-CD123-Luc.

FIGS. 5A-5B show cytotoxic activities of engineered T cells expressing various constructs against tumor cells: K562-CD123-Luc (FIG. 5A) and K562-Luc (FIG. 5B). “NKG2D-CD123 binder” designates engineered T cells expressing the LIC2004 dual chimeric receptor system. “NKG2D” designates engineered T cells expressing only the chimeric receptor comprising an NKG2D domain in the LIC2004 dual chimeric receptor system (i.e., LIC2004-1). “CD123 binder” designates engineered T cells expressing only the chimeric receptor comprising an IL-3 domain in the LIC2004 dual chimeric receptor system.

FIG. 6A shows blocking effects of MICA (a cognate ligand of NKG2D) on killing of K562-CD123-Luc and K562-Luc cells by engineered T cells expressing NKG2D×IL-3 chimeric receptor constructs. FIG. 6B shows that BSA has no significant blocking effects on killing of K562-CD123-Luc and K562-Luc cells by engineered T cells expressing NKG2D×IL-3 chimeric receptor constructs.

FIG. 7 shows cytotoxic activities of engineered T cells expressing LIC2004 and LIC2002-2 constructs against K562 and K562-CD123-Luc tumor cells.

FIG. 8A-8C show secreted levels of IFNγ by co-cultures of engineered T cells expressing LIC2002-2, LIC2004 and LIC2004-1 constructs with K562-CD123-Luc, K562-Luc and KG1-Luc cell lines.

### DETAILED DESCRIPTION OF THE PRESENT APPLICATION

The present application provides multispecific (e.g., bispecific) chimeric receptors and dual chimeric receptor systems that target NKG2D ligands and a second antigen such as CD123 (e.g., an IL-3 domain). In some embodiments, unlike antibody-based CARs, the chimeric receptors and dual chimeric receptor systems describe herein take advantage of high affinity and specificity between ligands and their cognate receptors on T cells. NKG2D ligands are expressed only on stressed cells, especially on tumor cells. Engineered immune cells expressing the NKG2D chimeric receptors and dual chimeric receptor systems described herein have enhanced antitumor capacity, and provide useful therapeutic agents for anti-cancer treatment.

Accordingly, one aspect of the present application provides a multispecific chimeric receptor comprising: (a) an extracellular domain comprising an NKG2D domain and a second antigen binding domain (e.g., a binding domain targeting CD123); (b) a transmembrane domain; and (c) an intracellular signaling domain.

In some embodiments, there is provided a multispecific chimeric receptor comprising a polypeptide chain comprising: (a) an extracellular domain comprising a second antigen binding domain (e.g., an IL-3 domain), a first NKG2D domain and a second NKG2D domain; (b) a transmembrane domain, and (c) an intracellular signaling domain.

In some embodiments, there is provided a multispecific chimeric receptor comprising a first polypeptide chain and a second polypeptide chain, each comprising: (a) an extracellular domain comprising an NKG2D domain and a second antigen binding domain (e.g., an IL-3 domain); (b) a transmembrane domain; and (c) an intracellular signaling domain. In some embodiments, the extracellular domain further comprises a dimerization motif, such as a leucine zipper.

In another aspect, there is provided a dual chimeric receptor system comprising: (i) a first chimeric receptor comprising (a) a first extracellular domain comprising an NKG2D domain; (b) a first transmembrane domain; and (c) a first intracellular signaling domain; (ii) a second chimeric receptor comprising (a) a second extracellular domain comprising a second antigen binding domain (e.g., an IL-3 domain); (b) a second transmembrane domain; and optionally (c) a second intracellular signaling domain. In some embodiments, the first chimeric receptor comprises a single polypeptide chain, wherein the first extracellular domain comprises a first NKG2D domain and a second NKG2D domain. In some embodiments, the first chimeric receptor comprises a first polypeptide chain and a second polypeptide chain, each comprising: (a) a first extracellular domain comprising an NKG2D domain; (b) a first transmembrane domain; and (c) a first intracellular signaling domain.

Engineered immune effector cells (such as T cells) comprising the chimeric receptors, multispecific chimeric receptors or dual chimeric receptor systems, pharmaceutical compositions, kits, articles of manufacture and methods of treating cancer using the engineered immune effector cells are also described herein.

### I. Definitions

“Chimeric receptor” as used herein refers to genetically engineered receptors, which can be used to graft one or more specific polypeptide interactions through antigen-antibody interaction or ligand-receptor binding onto immune effector cells, such as T cells. Some chimeric receptors are also known as “chimeric antigen receptors,” “artificial T-cell receptors,” “chimeric T cell receptors,” or “chimeric immune receptors.” In some embodiments, the chimeric receptor comprises an extracellular antigen binding domain specific for one or more antigens (such as tumor antigens), a transmembrane domain, and an intracellular signaling domain of a T cell and/or other receptors. In some embodiments, the extracellular antigen binding domain comprises at least one domain derived from a ligand or the extracellular domain of a receptor, wherein the ligand or receptor is a cell surface antigen, such as a tumor antigen.

“NKG2D chimeric receptor” refers to a chimeric receptor having an extracellular domain comprising one or more binding domains specific for NKG2D ligands (e.g., an NKG2D domain). “NKG2D×IL-3 chimeric receptor” refers to a chimeric receptor having an extracellular domain comprising a binding domain specific for NKG2D ligands (e.g., an NKG2D domain) and a binding domain specific for CD123 (e.g., an IL-3 domain).

As used herein, an “NKG2D domain” refers to a functional fragment in the extracellular domain of NKG2D, which can specifically bind to one or more NKG2D ligands upon dimerization of the NKG2D domain. Exemplary human NKG2D ligands include, but are not limited to, MICA, MICB, and ULBP molecules.

As used herein, an “IL-3 domain” refers to a functional fragment of IL-3 (including full-length IL-3), which can specifically bind to CD123, e.g., IL-3R complex and/or IL-3RA subunit.

As use herein, the term “targets,” “specifically binds,” “specifically recognizes,” or “is specific for” refers to measurable and reproducible interactions such as binding between a target and an antigen binding protein (such as an antigen binding domain, a ligand, or a chimeric receptor), which is determinative of the presence of the target in the presence of a heterogeneous population of molecules including biological molecules. For example, an antigen binding protein that specifically binds a target is an antigen binding protein that binds this target with greater affinity, avidity, more readily, and/or with greater duration than it binds other targets. In some embodiments, the extent of binding of an antigen binding protein to an unrelated target is less than about 10% of the binding of the antigen binding protein to the target as measured, e.g., by a radioimmunoassay (RIA). In some embodiments, an antigen binding protein that specifically binds a target has a dissociation constant (Kd) of ≤1 μM, ≤100 nM, ≤10 nM, ≤1 nM, or ≤0.1 nM. In some embodiments, an antigen binding protein specifically binds an epitope on a protein that is conserved among the protein from different species. In some embodiments, specific binding can include, but does not require exclusive binding.

The term “specificity” refers to selective recognition of an antigen binding protein (such as an antigen binding domain, a ligand or a chimeric receptor) for a particular epitope of an antigen. The term “multispecific” as used herein denotes that an antigen binding protein (such as a chimeric receptor) has two or more antigen-binding sites of which at least two bind different antigens. “Bispecific” as used herein denotes that an antigen binding protein (such as a chimeric receptor) has two different antigen-binding specificities.

“Binding affinity” generally refers to the strength of the sum total of non-covalent interactions between a single binding site of a molecule (such as an antigen binding domain, a ligand, or a chimeric receptor) and its binding partner (e.g., an antigen). Unless indicated otherwise, as used herein, “binding affinity” refers to intrinsic binding affinity that reflects a 1:1 interaction between members of a binding pair (e.g., an antigen binding domain and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present application.

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)₂, and Fv). Antibodies contemplated herein include single-domain antibodies, such as heavy chain only antibodies.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules; single-domain antibodies (such as V_{H}H), and multispecific antibodies formed from antibody fragments.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNAS TAR) software. Those skilled in the art can determine appropriate parameters for measuring alignment, including any algorithms needed to achieve maximal alignment over the full length of the sequences being compared.

An “isolated” nucleic acid molecule encoding a chimeric receptor or a dual chimeric receptor system described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the expressions “cell”, “cell line”, and “cell culture” are used interchangeably and all such designations include progeny. Thus, the words “transfectants” and “transfected cells” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present application contemplate any one or more of these aspects of treatment.

As used herein, an “individual” or a “subject” refers to a mammal, including, but not limited to, human, bovine, horse, feline, canine, rodent, or primate. In some embodiments, the individual is a human.

The term “effective amount” used herein refers to an amount of an agent, such as an engineered immune effector cell, or a pharmaceutical composition thereof, sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

It is understood that embodiments of the present application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

### II. Multispecific Chimeric Receptors and Dual Chimeric Receptor Systems

The present application provides chimeric receptors and chimeric receptor systems that target NKG2D ligands. One aspect of the present application provides a multispecific chimeric receptor comprising an extracellular domain comprising an NKG2D domain and a second antigen binding domain, such as a CD123 binding domain, e.g., an IL-3 domain.

In some embodiments, there is provided a chimeric receptor comprising: (a) an extracellular domain comprising an NKG2D domain; (b) a transmembrane domain; and (c) an intracellular signaling domain. In some embodiments, the transmembrane domain is derived from a molecule selected from the group consisting of CD8a, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the chimeric receptor further comprises a hinge region (such as a CD8a hinge region) located between the C-terminus of the extracellular domain and the N-terminus of the transmembrane domain. In some embodiments, the chimeric receptor comprises from the N-terminus to the C-terminus: a first NKG2D domain, a peptide linker, a second NKG2D domain, a transmembrane domain (CD8α), a co-stimulatory domain derived from 4-1BB, and a primary signaling domain derived from CD3ζ. In some embodiments, the NKG2D domain comprises the amino acid sequence of SEQ ID NO: 8.

In some embodiments, there is provided a chimeric receptor comprising a first polypeptide chain and a second polypeptide chain each comprising: (a) an extracellular domain comprising an NKG2D domain and a dimerization motif (e.g., a leucine zipper or cysteine zipper); (b) a transmembrane domain; and (c) an intracellular signaling domain. In some embodiments, the NKG2D domain of the first polypeptide chain is crosslinked to the NKG2D domain of the second polypeptide chain via one or more disulfide bonds. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, each polypeptide chain further comprises a hinge region (such as a CD8α hinge region) located between the C-terminus of the first extracellular domain and the N-terminus of the first transmembrane domain. In some embodiments, each polypeptide chain further comprises a signal peptide (such as a CD8α signal peptide) located at the N-terminus of each polypeptide chain.

In some embodiments, there is provided a chimeric receptor comprising: (a) an extracellular domain comprising a first NKG2D domain and a second NKG2D domain; (b) a transmembrane domain; and (c) an intracellular signaling domain. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the chimeric receptor further comprises a hinge region (such as a CD8α hinge region) located between the C-terminus of the extracellular domain and the N-terminus of the transmembrane domain. In some embodiments, the chimeric receptor comprises from the N-terminus to the C-terminus: a first NKG2D domain, a peptide linker, a second NKG2D domain, a transmembrane domain (CD8α), a co-stimulatory domain derived from 4-1BB, and a primary signaling domain derived from CD3ζ. In some embodiments, the NKG2D domain comprises the amino acid sequence of SEQ ID NO: 8. In some embodiments, there is provided a chimeric receptor comprising an amino acid sequence having at least about any one of 85%, 86%, 87%, 88%, 89%, 90%, 91%, 92%, 93%, 94%, 95%, 96%, 97%, 98%, 99%, or 100% sequence identity to an amino acid sequence to SEQ ID NO: 33. In some embodiments, there is provided an isolated nucleic acid sequence comprising a nucleic acid sequence having at least about any one of 85%, 86%, 87%, 88%, 89%, 90%, 91%, 92%, 93%, 94%, 95%, 96%, 97%, 98%, 99%, or 100% sequence identity to a nucleic acid sequence of SEQ ID NO: 38.

In some embodiments, there is provided a multispecific (e.g., bispecific) chimeric receptor comprising: (a) an extracellular domain comprising an NKG2D domain and a second antigen binding domain (e.g., an IL-3 domain); (b) a transmembrane domain; and (c) an intracellular signaling domain. In some embodiments, the second antigen binding domain specifically binds to an antigen selected from the group consisting of CD19, CD20, CD22, CD33, CD38, BCMA, CS1, ROR1, GPC3, CD123, IL-13R, CD138, c-Met, EGFR, EGFRvIII, HER2, HER3, GD-2, NY-ESO-1, MAGE A3, and glycolipid F77. In some embodiments, the second antigen binding domain is a ligand or a ligand binding domain derived from a molecule selected from the group consisting of NKG2A, NKG2C, NKG2F, IL-3, IL-13, LLT1, AICL, DNAM-1, and NKp80. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the multispecific chimeric receptor further comprises a hinge region (such as a CD8a hinge region) located between the C-terminus of the extracellular domain and the N-terminus of the transmembrane domain.

The chimeric receptor (e.g., multispecific chimeric receptor) may comprise one or more polypeptide chains. In some embodiments, the chimeric receptor is monomeric. In some embodiments, the monomeric chimeric receptor comprises an extracellular domain comprising a first NKG2D domain and a second NKG2D domain. In some embodiments, the extracellular domain comprises from the N-terminus to the C-terminus: the second antigen binding domain (e.g., an IL-3 domain), the first NKG2D domain and the second NKG2D domain. In some embodiments, the extracellular domain comprises from the N-terminus to the C-terminus: the first NKG2D domain, the second NKG2D domain, and the second antigen binding domain (e.g., an IL-3 domain). In some embodiments, the first NKG2D domain is crosslinked to the second NKG2D domain. In some embodiments, the first NKG2D domain comprises a first engineered residue at the N-terminus, and the second NKG2D domain comprises a second engineered residue at the C-terminus, wherein the first engineered residue is associates with the second engineered residue, e.g., via a disulfide bond, or a salt bridge. In some embodiments, the second antigen binding domain is fused to the first NKG2D domain via a peptide linker, such as a peptide linker of no more than about 50 amino acids long, e.g., a peptide linker comprising an amino acid sequence selected from SEQ ID NOs: 12-15.

In some embodiments, the chimeric receptor (e.g., multispecific chimeric receptor) is dimeric, such as homodimeric or heterodimeric. In some embodiments, the dimeric chimeric receptor comprises two polypeptide chains each comprising a single NKG2D domain. In some embodiments, the dimeric chimeric receptor comprises two identical polypeptide chains. In some embodiments, the dimeric chimeric receptor comprises two different polypeptide chains. In some embodiments, each polypeptide chain comprises from the N-terminus to the C terminus: the second antigen binding domain, the NKG2D domain, the transmembrane domain, and the intracellular signaling domain. In some embodiments, each polypeptide chain comprises from the N-terminus to the C terminus: the NKG2D domain, the second antigen binding domain, the transmembrane domain, and the intracellular signaling domain. In some embodiments, the NKG2D domain of each polypeptide chain of a dimeric chimeric receptor associate non-covalently with each other to form a dimer. In some embodiments, the NKG2D domain of each polypeptide chain of a dimeric chimeric receptor associates covalently with each other to form a dimer, e.g., by disulfide bonds, and/or via dimerization motifs (e.g., leucine zippers, or cysteine zippers) in the extracellular domain. In some embodiments, the second antigen binding domain is fused to the NKG2D domain via a peptide linker, such as a peptide linker of no more than about 50 amino acids long, e.g., a peptide linker comprising an amino acid sequence selected from SEQ ID NOs: 12-15. In some embodiments, the second antigen binding domain is fused to the NKG2D domain via a dimerization motif, such as a leucine zipper or a cysteine zipper.

Thus, in some embodiments, there is provided a multispecific (e.g., bispecific) chimeric receptor comprising a polypeptide chain comprising: (a) an extracellular domain comprising a first NKG2D domain, a second NKG2D domain, and a second antigen binding domain; (b) a transmembrane domain; and (c) an intracellular signaling domain. In some embodiments, the second antigen binding domain specifically binds to an antigen selected from the group consisting of CD19, CD20, CD22, CD33, CD38, BCMA, CS1, ROR1, GPC3, CD123, IL-13R, CD138, c-Met, EGFR, EGFRvIII, HER2, HER3, GD-2, NY-ESO-1, MAGE A3, and glycolipid F77. In some embodiments, the second antigen binding domain is a ligand or a ligand binding domain derived from a molecule selected from the group consisting of NKG2A, NKG2C, NKG2F, IL-3, IL-13, LLT1, AICL, DNAM-1, and NKp80. In some embodiments, the second antigen binding domain is fused to the first NKG2D domain or the second NKG2D domain via a peptide linker, such as a peptide linker of no more than about 50 amino acids long, e.g., a peptide linker comprising an amino acid sequence selected from SEQ ID NOs: 12-15. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the multispecific chimeric receptor further comprises a hinge region (such as a CD8a hinge region) located between the C-terminus of the extracellular domain and the N-terminus of the transmembrane domain. In some embodiments, the multispecific chimeric receptor further comprises a signal peptide (such as a CD8a signal peptide) located at the N-terminus of the polypeptide. In some embodiments, the polypeptide chain comprises from the N-terminus to the C-terminus: the second antigen binding domain (e.g., an IL-3 domain), a first peptide linker, the first NKG2D domain, a second peptide linker, the second NKG2D domain, a CD8a hinge region, a CD8a transmembrane domain, a co-stimulatory signaling domain derived from 4-1BB, and a primary intracellular signaling domain derived from CD3ζ.

In some embodiments, there is provided a multispecific (e.g., bispecific) chimeric receptor comprising a first polypeptide chain and a second polypeptide chain each comprising: (a) an extracellular domain comprising an NKG2D domain and a second antigen binding domain; (b) a transmembrane domain; and (c) an intracellular signaling domain. In some embodiments, the NKG2D domain of the first polypeptide chain is crosslinked to the NKG2D domain of the second polypeptide chain via one or more disulfide bonds. In some embodiments, the second antigen binding domain specifically binds to an antigen selected from the group consisting of CD19, CD20, CD22, CD33, CD38, BCMA, CS1, ROR1, GPC3, CD123, IL-13R, CD138, c-Met, EGFR, EGFRvIII, HER2, HER3, GD-2, NY-ESO-1, MAGE A3, and glycolipid F77. In some embodiments, the second antigen binding domain is a ligand or a ligand binding domain derived from a molecule selected from the group consisting of NKG2A, NKG2C, NKG2F, IL-3, IL-13, LLT1, AICL, DNAM-1, and NKp80. In some embodiments, the second antigen binding domain is fused to the NKG2D domain via a peptide linker, such as a peptide linker of no more than about 50 amino acids long, e.g., a peptide linker comprising an amino acid sequence selected from SEQ ID NOs: 12-15. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, each polypeptide chain further comprises a hinge region (such as a CD8a hinge region) located between the C-terminus of the first extracellular domain and the N-terminus of the first transmembrane domain. In some embodiments, each polypeptide chain further comprises a signal peptide (such as a CD8a signal peptide) located at the N-terminus of each polypeptide chain. In some embodiments, each of the first polypeptide chain and the second polypeptide chain comprises from the N-terminus to the C-terminus: the second antigen binding domain (e.g., an IL-3 domain), a peptide linker, the NKG2D domain, a CD8a hinge region, a CD8α transmembrane domain, a co-stimulatory signaling domain derived from 4-1BB, and a primary intracellular signaling domain derived from CD3ζ.

In some embodiments, there is provided a multispecific (e.g., bispecific) chimeric receptor comprising a first polypeptide chain and a second polypeptide chain each comprising: (a) an extracellular domain comprising an NKG2D domain, a dimerization motif (e.g., a leucine zipper or cysteine zipper), and a second antigen binding domain; (b) a transmembrane domain; and (c) an intracellular signaling domain. In some embodiments, the NKG2D domain of the first polypeptide chain is crosslinked to the NKG2D domain of the second polypeptide chain via one or more disulfide bonds. In some embodiments, the second antigen binding domain specifically binds to an antigen selected from the group consisting of CD19, CD20, CD22, CD33, CD38, BCMA, CS1, ROR1, GPC3, CD123, IL-13R, CD138, c-Met, EGFR, EGFRvIII, HER2, HER3, GD-2, NY-ESO-1, MAGE A3, and glycolipid F77. In some embodiments, the second antigen binding domain is a ligand or a ligand binding domain derived from a molecule selected from the group consisting of NKG2A, NKG2C, NKG2F, IL-3, IL-13, LLT1, AICL, DNAM-1, and NKp80. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, each polypeptide chain further comprises a hinge region (such as a CD8α hinge region) located between the C-terminus of the first extracellular domain and the N-terminus of the first transmembrane domain. In some embodiments, each polypeptide chain further comprises a signal peptide (such as a CD8α signal peptide) located at the N-terminus of each polypeptide chain. In some embodiments, each of the first polypeptide chain and the second polypeptide chain comprises from the N-terminus to the C-terminus: the second antigen binding domain (e.g., an IL-3 domain), a leucine zipper, the NKG2D domain, a CD8α hinge region, a CD8α transmembrane domain, a co-stimulatory signaling domain derived from 4-1BB, and a primary intracellular signaling domain derived from CD3ζ.

In some embodiments, the second antigen binding domain specifically binds to a cell surface antigen, such as a tumor antigen. Exemplary tumor antigens include, but are not limited to CD19, CD20, CD22, CD33, CD38, BCMA, CS1, ROR1, GPC3, CD123, CD138, c-Met, EGFR, EGFRvIII, HER2, HER3, GD-2, NY-ESO-1, MAGE A3, and glycolipid F77. In some embodiments, the second antigen binding domain is an antibody fragment, such as a single-chain antibody (e.g., scFv) or a single-domain antibody (e.g., a VHH). In some embodiments, the second antigen binding domain is an antibody fragment (e.g. scFv or VHH) that specifically binds to CD123 (e.g., IL-3R or IL-3RA subunit).

In some embodiments, the second antigen binding domain is a ligand. In some embodiments, the second antigen binding domain is a ligand-binding domain, such as an extracellular domain of a receptor. Exemplary ligands and receptors include, but are not limited to, NKG2A, NKG2C, NKG2F, IL-3, IL-13, LLT1, AICL, DNAM-1, and NKp80. In some embodiments, the second antigen binding domain is an IL-3 domain.

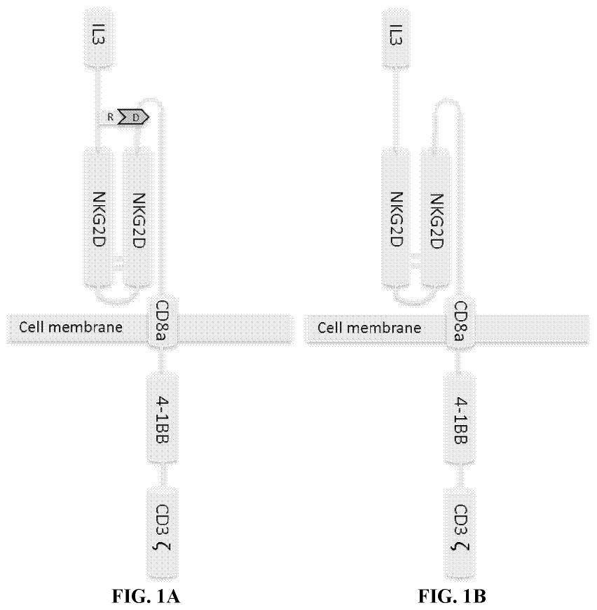

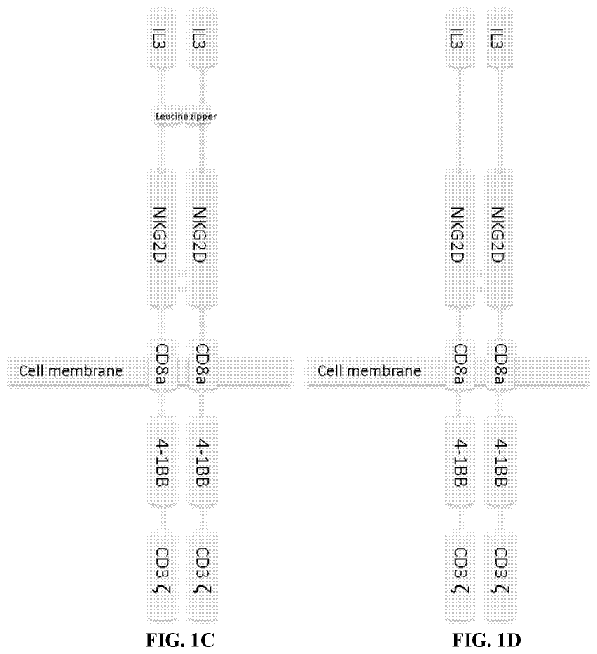

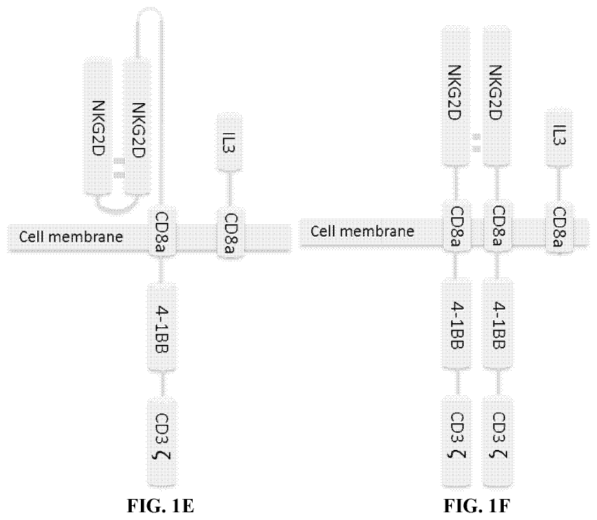

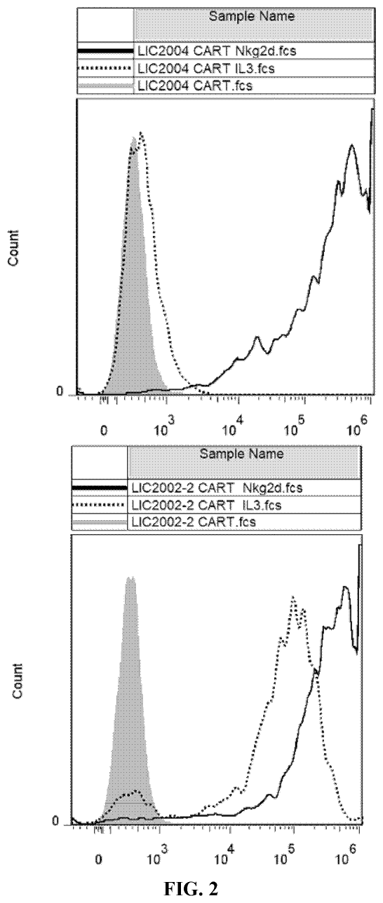

Thus, in some embodiments, there is provided a multispecific (e.g., bispecific) chimeric receptor comprising a polypeptide chain comprising: (a) an extracellular domain comprising a first NKG2D domain, a second NKG2D domain, and a CD123 binding domain (e.g., an IL-3 domain); (b) a transmembrane domain; and (c) an intracellular signaling domain. In some embodiments, the CD123 binding domain is fused to the first NKG2D domain or the second NKG2D domain via a peptide linker, such as a peptide linker of no more than about 50 amino acids long, e.g., a peptide linker comprising an amino acid sequence selected from SEQ ID NOs: 12-15. In some embodiments, the CD123 binding domain is an anti-CD123 antibody fragment (e.g., scFv or VHH). In some embodiments, the CD123 binding domain is an IL-3 domain. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the multispecific chimeric receptor further comprises a hinge region (such as a CD8α hinge region) located between the C-terminus of the extracellular domain and the N-terminus of the transmembrane domain. In some embodiments, the multispecific chimeric receptor further comprises a signal peptide (such as a CD8α signal peptide) located at the N-terminus of the polypeptide. In some embodiments, the polypeptide chain comprises from the N-terminus to the C-terminus: an IL-3 domain, a first peptide linker, the first NKG2D domain, a second peptide linker, the second NKG2D domain, a CD8α hinge region, a CD8α transmembrane domain, a co-stimulatory signaling domain derived from 4-1BB, and a primary intracellular signaling domain derived from CD3ζ. Exemplary multispecific chimeric receptors are shown in FIGS. 1A-1B.

In some embodiments, there is provided a multispecific (e.g., bispecific) chimeric receptor comprising a first polypeptide chain and a second polypeptide chain each comprising: (a) an extracellular domain comprising an NKG2D domain and a CD123 binding domain (e.g., an IL-3 domain); (b) a transmembrane domain; and (c) an intracellular signaling domain. In some embodiments, the NKG2D domain of the first polypeptide chain is crosslinked to the NKG2D domain of the second polypeptide chain via one or more disulfide bonds. In some embodiments, the CD123 binding domain is fused to the NKG2D domain via a peptide linker, such as a peptide linker of no more than about 50 amino acids long, e.g., a peptide linker comprising an amino acid sequence selected from SEQ ID NOs: 12-15. In some embodiments, the CD123 binding domain is an anti-CD123 antibody fragment (e.g., scFv or VHH). In some embodiments, the CD123 binding domain is an IL-3 domain. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, each polypeptide chain further comprises a hinge region (such as a CD8α hinge region) located between the C-terminus of the first extracellular domain and the N-terminus of the first transmembrane domain. In some embodiments, each polypeptide chain further comprises a signal peptide (such as a CD8α signal peptide) located at the N-terminus of each polypeptide chain. In some embodiments, each of the first polypeptide chain and the second polypeptide chain comprises from the N-terminus to the C-terminus: an IL-3 domain, a peptide linker, the NKG2D domain, a CD8α hinge region, a CD8α transmembrane domain, a co-stimulatory signaling domain derived from 4-1BB, and a primary intracellular signaling domain derived from CD3ζ. An exemplary multispecific chimeric receptor is shown in FIG. 1D.

In some embodiments, there is provided a multispecific (e.g., bispecific) chimeric receptor comprising a first polypeptide chain and a second polypeptide chain each comprising: (a) an extracellular domain comprising an NKG2D domain, a dimerization motif (e.g., a leucine zipper, or cysteine zipper), and a CD123 binding domain (e.g., an IL-3 domain); (b) a transmembrane domain; and (c) an intracellular signaling domain. In some embodiments, the NKG2D domain of the first polypeptide chain is crosslinked to the NKG2D domain of the second polypeptide chain via one or more disulfide bonds. In some embodiments, the CD123 binding domain is an anti-CD123 antibody fragment (e.g., scFv or VHH). In some embodiments, the CD123 binding domain is an IL-3 domain. In some embodiments, the transmembrane domain is derived from a molecule selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, each polypeptide chain further comprises a hinge region (such as a CD8α hinge region) located between the C-terminus of the first extracellular domain and the N-terminus of the first transmembrane domain. In some embodiments, each polypeptide chain further comprises a signal peptide (such as a CD8α signal peptide) located at the N-terminus of each polypeptide chain. In some embodiments, each of the first polypeptide chain and the second polypeptide chain comprises from the N-terminus to the C-terminus: an IL-3 domain, a leucine zipper, the NKG2D domain, a CD8α hinge region, a CD8α transmembrane domain, a co-stimulatory signaling domain derived from 4-1BB, and a primary intracellular signaling domain derived from CD3ζ. An exemplary multispecific chimeric receptor is shown in FIG. 1C.

Exemplary NKG2D×IL-3 chimeric receptors and their sequences are shown in Table 1. For dimeric chimeric receptors having two identical polypeptide chains, the amino acid sequences of the monomeric subunits are shown. In some embodiments, there is provided an NKG2D×IL-3 chimeric receptor comprising a polypeptide having at least about any one of 85%, 86%, 87%, 88%, 89%, 90%, 91%, 92%, 93%, 94%, 95%, 96%, 97%, 98%, 99%, or 100% sequence identity to an amino acid sequence selected from the group consisting of SEQ ID NOs: 16-20. In some embodiments, there is provided an NKG2D×IL-3 chimeric receptor comprising an amino acid sequence selected from the group consisting of SEQ ID NOs: 16-20. In some embodiments, there is provided an NKG2D chimeric receptor comprising a polypeptide having at least about any one of 85%, 86%, 87%, 88%, 89%, 90%, 91%, 92%, 93%, 94%, 95%, 96%, 97%, 98%, 99%, or 100% sequence identity to the amino acid sequence of SEQ ID NO: 33. In some embodiments, there is provided an NKG2D chimeric receptor comprising the amino acid sequence of SEQ ID NO: 33. Also provided is a polypeptide comprising an amino acid sequence selected from the group consisting of SEQ ID NOs: 16-20 and 33.

In some embodiments, there is provided one or more isolated nucleic acids encoding any of the chimeric receptors or multispecific chimeric receptors provided herein. In some embodiments, wherein the chimeric receptor is a dimeric chimeric receptor having two identical polypeptide chains, the isolated nucleic acid encoding a monomeric subunit of the chimeric receptor, i.e., a single copy of the polypeptide chain, is provided. In some embodiments, there is provided an isolated nucleic acid having at least about any one of 85%, 86%, 87%, 88%, 89%, 90%, 91%, 92%, 93%, 94%, 95%, 96%, 97%, 98%, 99%, or 100% sequence identity to a nucleic acid sequence selected from the group consisting of SEQ ID NOs: 21-25 and 38. In some embodiments, the isolated nucleic acid is a DNA. In some embodiments, the isolated nucleic acid is an RNA (e.g., mRNA). In some embodiments, there is provided one or more vectors comprising any one of the nucleic acids encoding the chimeric receptors or multispecific chimeric receptors described above. In some embodiments, the vector is an expression vector. In some embodiments, the vector is a viral vector, such as a lentiviral vector. In some embodiments, the vector is a non-viral vector.

TABLE 1
Exemplary NKG2D × IL-3 chimeric receptors.

| | | | Extracellular | | | | | | |
|---|---|---|---|---|---|---|---|---|---|
| | | | domain | | | | | Intracellular | |
| Chimeric | AA | NA | Domain | Domain | Domain | | | signaling | |
| Receptor | SEQ ID | SEQ ID | 1 | 2 | 3 | Hinge | TM | CO2 | Prim. |
| LIC2001 | 16 | 21 | IL-3 | RR- | Forward | CD8α | CD8α | 4-1BB | CD3ζ |
| | | | | reverse | NKG2D- | | | | |
| | | | | NKG2D | DD | | | | |
| LIC2001-1 | 17 | 22 | IL-3 | Reverse | Forward | CD8α | CD8α | 4-1BB | CD3ζ |
| | | | | NKG2D | NKG2D | | | | |
| LIC2002 | 18 | 23 | IL-3 | Leucine | Reverse | CD8α | CD8α | 4-1BB | CD3ζ |
| | | | | zipper | NKG2D | | | | |
| LIC2002-1 | 19 | 24 | IL-3 | — | Reverse | CD8α | CD8α | CD137 | CD3ζ |
| | | | | | NKG2D | | | | |
| LIC2002-2 | 20 | 25 | IL-3 | Leucine | Forward | CD8α | CD8α | CD137 | CD3ζ |
| | | | | zipper | NKG2D | | | | |

### Dual Chimeric Receptor System

One aspect of the present application provides a dual chimeric receptor system comprising: (i) a first chimeric receptor comprising: (a) a first extracellular domain comprising an NKG2D domain, (b) a first transmembrane domain, and (c) a first intracellular signaling domain; and (ii) a second chimeric receptor comprising: (a) a second extracellular domain comprising a second antigen binding domain, (b) a second transmembrane domain, and optionally (c) a second intracellular signaling domain. The first chimeric receptor specifically binds to NKG2D ligands, and the second chimeric receptor specifically binds to a second antigen, such as a tumor antigen, e.g., CD123.

Each of the first chimeric receptor and the second chimeric receptor may comprise one or more polypeptide chains. The dual chimeric receptor system may comprise any combination of the first chimeric receptor and the second chimeric receptor described herein.

In some embodiments, the first chimeric receptor comprises a single polypeptide chain comprising: (a) a first extracellular domain comprising a first NKG2D domain and a second NKG2D domain, (b) a first transmembrane domain, and (c) a first intracellular signaling domain. In some embodiments, the first NKG2D domain is crosslinked to the second NKG2D domain. In some embodiments, the first NKG2D domain comprises a first engineered residue at the N-terminus, and the second NKG2D domain comprises a second engineered residue at the C-terminus, wherein the first engineered residue is associates with the second engineered residue, e.g., via a disulfide bond, or a salt bridge. In some embodiments, the first transmembrane domain is derived from a molecule selected from the group consisting of CD8α, CD4, CD28, CD137, CD80, CD86, CD152 and PD1. In some embodiments, the first intracellular signaling domain comprises a primary intracellular signaling domain of an immune effector cell (such as T cell). In some embodiments, the primary intracellular signaling domain is derived from CD3ζ. In some embodiments, the first intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the first chimeric receptor further comprises a hinge region (such as a CD8α hinge region) located between the C-terminus of the first extracellular domain and the N-terminus of the first transmembrane domain. In some embodiments, the first chimeric receptor further comprises a signal peptide (such as a CD8α signal peptide) located at the N-terminus of the polypeptide chain. In some embodiments, the first chimeric receptor comprises a polypeptide chain comprising from the N-terminus to the C-terminus: a first NKG2D domain, a peptide linker, a second NKG2D domain, a CD8α hinge region, a CD8α transmembrane domain, a co-stimulatory signaling domain derived from 4-1BB, and a primary intracellular signaling domain derived from CD3ζ. An exemplary first chimeric receptor is shown in FIG. 1E.

In some embodiments, the first chimeric receptor comprises a first polypeptide chain and a second polypeptide chain each comprising: (a) a first extracellular domain comprising an NKG2D domain, (b) a first transmembrane domain, and (c) a first intracellular signaling domain. In some embodiments, the NKG2D domain of the first polypeptide chain is crosslinked to the NKG2D domain of the second polypeptide chain via one or more disulfide bonds. In some embodiments, the first transmembrane domain is derived from a molecule selected from the group consisting of CD8α, CD4, CD28, CD137, CD80, CD86, CD152 and PD1. In some embodiments, the first intracellular signaling domain comprises a primary intracellular signaling domain of an immune effector cell (such as T cell). In some embodiments, the primary intracellular signaling domain is derived from CD3ζ. In some embodiments, the first intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, each polypeptide chain further comprises a hinge region (such as a CD8α hinge region) located between the C-terminus of the first extracellular domain and the N-terminus of the first transmembrane domain. In some embodiments, each polypeptide chain further comprises a signal peptide (such as a CD8α signal peptide) located at the N-terminus of each polypeptide chain. In some embodiments, each of the first polypeptide chain and the second polypeptide chain comprises from the N-terminus to the C-terminus: the NKG2D domain, a CD8α hinge region, a CD8α transmembrane domain, a co-stimulatory signaling domain derived from 4-1BB, and a primary intracellular signaling domain derived from CD3ζ. An exemplary first chimeric receptor is shown in FIG. 1F.

In some embodiments, the first chimeric receptor comprises a first polypeptide chain and a second polypeptide chain each comprising: (a) a first extracellular domain comprising an NKG2D domain and a dimerization domain (e.g., a leucine zipper, or cysteine zipper), (b) a first transmembrane domain, and (c) a first intracellular signaling domain. In some embodiments, the dimerization domain is located at the N-terminus of the NKG2D domain. In some embodiments, the dimerization domain is located at the C-terminus of the NKG2D domain. In some embodiments, the NKG2D domain of the first polypeptide chain is further crosslinked to the NKG2D domain of the second polypeptide chain via one or more disulfide bonds. In some embodiments, the first transmembrane domain is derived from a molecule selected from the group consisting of CD8α, CD4, CD28, CD137, CD80, CD86, CD152 and PD1. In some embodiments, the first intracellular signaling domain comprises a primary intracellular signaling domain of an immune effector cell (such as T cell). In some embodiments, the primary intracellular signaling domain is derived from CD3ζ. In some embodiments, the first intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, each polypeptide chain further comprises a hinge region (such as a CD8α hinge region) located between the C-terminus of the first extracellular domain and the N-terminus of the first transmembrane domain. In some embodiments, each polypeptide chain further comprises a signal peptide (such as a CD8α signal peptide) located at the N-terminus of each polypeptide chain. In some embodiments, each of the first polypeptide chain and the second polypeptide chain comprises from the N-terminus to the C-terminus: a leucine zipper, the NKG2D domain, a CD8α hinge region, a CD8α transmembrane domain, a co-stimulatory signaling domain derived from 4-1BB, and a primary intracellular signaling domain derived from CD3.

In some embodiments, the second chimeric receptor comprises a polypeptide chain comprising: (a) a second extracellular domain comprising a second antigen binding domain, and (b) a second transmembrane domain. In some embodiments, the second chimeric receptor does not comprise an intracellular signaling domain. In some embodiments, the second extracellular domain further comprises an NKG2D domain, for example, the second extracellular domain comprises from the N-terminus to the C-terminus: an NKG2D domain and a second antigen binding domain, or a second antigen binding domain and an NKG2D domain. In some embodiments, the second antigen binding domain specifically binds to an antigen selected from the group consisting of CD19, CD20, CD22, CD33, CD38, BCMA, CS1, ROR1, GPC3, CD123, IL-13R, CD138, c-Met, EGFR, EGFRvIII, HER2, HERS, GD-2, NY-ESO-1, MAGE A3, and glycolipid F77. In some embodiments, the second antigen binding domain is a ligand or a ligand binding domain derived from a molecule selected from the group consisting of NKG2A, NKG2C, NKG2F, IL-3, IL-13, LLT1, AICL, DNAM-1, and NKp80. In some embodiments, the second antigen binding domain is a CD123 binding domain, such as an IL-3 domain. In some embodiments, the second transmembrane domain is derived from a molecule selected from the group consisting of CD8α, CD4, CD28, CD137, CD80, CD86, CD152 and PD1. In some embodiments, the second chimeric receptor further comprises a hinge region (such as a CD8α hinge region) located between the C-terminus of the second extracellular domain and the N-terminus of the second transmembrane domain. In some embodiments, the second chimeric receptor further comprises a signal peptide (such as a CD8α signal peptide) located at the N-terminus of the polypeptide chain. In some embodiments, the second chimeric receptor comprises a polypeptide comprising from the N-terminus to the C-terminus: an IL-3 domain, a CD8α hinge region, and a CD8α transmembrane domain. An exemplary second chimeric receptor is shown in FIG. 1E and FIG. 1F.

In some embodiments, the second chimeric receptor comprises a polypeptide chain comprising: (a) a second extracellular domain comprising a second antigen binding domain, (b) a second transmembrane domain, and (c) a second intracellular signaling domain. In some embodiments, the second extracellular domain further comprises an NKG2D domain, for example, the second extracellular domain comprises from the N-terminus to the C-terminus: an NKG2D domain and a second antigen binding domain, or a second antigen binding domain and an NKG2D domain. In some embodiments, the second antigen binding domain specifically binds to an antigen selected from the group consisting of CD19, CD20, CD22, CD33, CD38, BCMA, CS1, ROR1, GPC3, CD123, IL-13R, CD138, c-Met, EGFR, EGFRvIII, HER2, HER3, GD-2, NY-ESO-1, MAGE A3, and glycolipid F77. In some embodiments, the second antigen binding domain is a ligand or a ligand binding domain derived from a molecule selected from the group consisting of NKG2A, NKG2C, NKG2F, IL-3, IL-13, LLT1, AICL, DNAM-1, and NKp80. In some embodiments, the second antigen binding domain is a CD123 binding domain, such as an IL-3 domain. In some embodiments, the second transmembrane domain is derived from a molecule selected from the group consisting of CD8α, CD4, CD28, CD137, CD80, CD86, CD152 and PD1. In some embodiments, the second intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the second intracellular signaling domain comprises a primary intracellular signaling domain of an immune effector cell (such as T cell). In some embodiments, the primary intracellular signaling domain is derived from CD3ζ. In some embodiments, the second intracellular signaling domain does not comprise a primary intracellular signaling domain. In some embodiments, the second chimeric receptor further comprises a hinge region (such as a CD8α hinge region) located between the C-terminus of the extracellular domain and the N-terminus of the transmembrane domain. In some embodiments, the second chimeric receptor further comprises a signal peptide (such as a CD8α signal peptide) located at the N-terminus of the polypeptide chain. In some embodiments, the second chimeric receptor comprises a polypeptide comprising from the N-terminus to the C-terminus: an IL-3 domain, a CD8α hinge region, a CD8α transmembrane domain, and a co-stimulatory signaling domain derived from 4-1BB.

In some embodiments, there is provided a dual chimeric receptor system comprising: (i) a first chimeric receptor comprising a first polypeptide chain and a second polypeptide chain each comprising: (a) a first extracellular domain comprising an NKG2D domain, (b) a first transmembrane domain, and (c) a first intracellular signaling domain; and (ii) a second chimeric receptor comprising a third polypeptide chain comprising: (a) a second extracellular domain comprising a second antigen binding domain (e.g., an IL-3 domain), (b) a second transmembrane domain, and optionally (c) a second intracellular signaling domain. In some embodiments, the NKG2D domain of the first polypeptide chain is crosslinked to the NKG2D domain of the second polypeptide chain via one or more disulfide bonds. In some embodiments, the first transmembrane domain and/or the second transmembrane domain is derived from a molecule selected from the group consisting of CD8α, CD4, CD28, CD137, CD80, CD86, CD152 and PD1. In some embodiments, the first intracellular signaling domain comprises a primary intracellular signaling domain of an immune effector cell (such as T cell). In some embodiments, the primary intracellular signaling domain is derived from CD3ζ. In some embodiments, the first intracellular signaling domain and/or the second intracellular domain comprises a co-stimulatory signaling domain. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the second chimeric receptor does not comprise an intracellular signaling domain. In some embodiments, each polypeptide chain further comprises a hinge region (such as a CD8α hinge region) located between the C-terminus of the first extracellular domain and the N-terminus of the first transmembrane domain. In some embodiments, each polypeptide chain further comprises a signal peptide (such as a CD8α signal peptide) located at the N-terminus of each polypeptide chain. In some embodiments, each of the first polypeptide chain and the second polypeptide chain comprises from the N-terminus to the C-terminus: the NKG2D domain, a CD8α hinge region, a CD8α transmembrane domain, a co-stimulatory signaling domain derived from 4-1BB, and a primary intracellular signaling domain derived from CD3ζ. In some embodiments, the third polypeptide chain comprises from the N-terminus to the C-terminus: an IL-3 domain and a CD8α transmembrane domain. An exemplary dual chimeric receptor system is shown in FIG. 1F.

In some embodiments, the polypeptide(s) of the first chimeric receptor and the polypeptide(s) of the second chimeric receptor are expressed via a polycistronic nucleic acid construct. For example, the polypeptide(s) of the first chimeric receptor is fused to the polypeptide(s) of the second chimeric receptor via a self-cleaving peptide. Exemplary self-cleaving peptides include, but are not limited to, T2A, P2A, and F2A peptide. The T2A peptide has been described, for example, see Szymczak A L. et al., Correction of multi-gene deficiency in vivo using a “self-cleaving” 2A peptide-based retroviral vector. Nat Biotechnol 2004; 22(5)589-594.

In some embodiments, there is provided one or more isolated nucleic acids encoding any one of the dual chimeric receptor systems described herein. In some embodiments, there is provided an isolated nucleic acid comprising a first nucleic acid sequence encoding the first chimeric receptor and a second nucleic acid sequence encoding the second chimeric receptor, wherein the first nucleic acid sequence is operably linked to the second nucleic acid sequence via a third nucleic acid sequence encoding a self-cleaving peptide (e.g., T2A). In some embodiments, wherein the first chimeric receptor is a dimeric chimeric receptor having two identical polypeptide chains, the first nucleic acid encodes a monomeric subunit of the first chimeric receptor, i.e., a single copy of the polypeptide chain.

In some embodiments, there is provided a chimeric receptor comprising an amino acid sequence having at least about any one of 85%, 86%, 87%, 88%, 89%, 90%, 91%, 92%, 93%, 94%, 95%, 96%, 97%, 98%, 99%, or 100% sequence identity to an amino acid sequence selected from the group consisting of SEQ ID NOs: 34-35 and 41-42. In some embodiments, there is provided an NKG2D×IL-3 dual chimeric receptor system comprising a first chimeric receptor comprising an amino acid sequence having at least about any one of 85%, 86%, 87%, 88%, 89%, 90%, 91%, 92%, 93%, 94%, 95%, 96%, 97%, 98%, 99%, or 100% sequence identity to the amino acid sequence of SEQ ID NO: 34, and a second chimeric receptor comprising an amino acid sequence having at least about any one of 85%, 86%, 87%, 88%, 89%, 90%, 91%, 92%, 93%, 94%, 95%, 96%, 97%, 98%, 99%, or 100% sequence identity to the amino acid sequence of SEQ ID NO: 41. In some embodiments, there is provided an NKG2D×IL-3 dual chimeric receptor system comprising a first chimeric receptor comprising an amino acid sequence having at least about any one of 85%, 86%, 87%, 88%, 89%, 90%, 91%, 92%, 93%, 94%, 95%, 96%, 97%, 98%, 99%, or 100% sequence identity to the amino acid sequence of SEQ ID NO: 35, and a second chimeric receptor comprising an amino acid sequence having at least about any one of 85%, 86%, 87%, 88%, 89%, 90%, 91%, 92%, 93%, 94%, 95%, 96%, 97%, 98%, 99%, or 100% sequence identity to the amino acid sequence of SEQ ID NO: 42. In some embodiments, there is provided a polypeptide comprising an amino acid sequence having at least about any one of 85%, 86%, 87%, 88%, 89%, 90%, 91%, 92%, 93%, 94%, 95%, 96%, 97%, 98%, 99%, or 100% sequence identity to the amino acid sequence of SEQ ID NO: 36 or 37. In some embodiments, there is provided an isolated nucleic acid having at least about any one of 85%, 86%, 87%, 88%, 89%, 90%, 91%, 92%, 93%, 94%, 95%, 96%, 97%, 98%, 99%, or 100% sequence identity to a nucleic acid sequence selected from the group consisting of SEQ ID NOs: 26-27, 39-40 and 43-44. In some embodiments, the isolated nucleic acid is a DNA. In some embodiments, the isolated nucleic acid is an RNA (e.g., an mRNA). In some embodiments, there is provided one or more vectors comprising any one or more of the nucleic acids encoding the dual chimeric receptor systems or chimeric receptors described above. In some embodiments, the vector is an expression vector. In some embodiments, the vector is a viral vector, such as a lentiviral vector. In some embodiments, the vector is a non-viral vector. An exemplary dual chimeric receptor system is shown below:

TABLE 2
Exemplary NKG2D × IL-3 dual chimeric receptor systems.

| | AA | NA | | Self- | |
|---|---|---|---|---|---|
| Chimeric | SEQ | SEQ | | cleaving | |
| Receptor | ID | ID | Chimeric receptor 1 | peptide | Chimeric receptor 2 |
| LIC2003 | 36 | 26 | Reverse NKG2D-forward | T2A | IL3-CD8α |
| | | | NKG2D-CD8α-4-1BB-CD3ζ | | (LIC2003-2: AA SEQ ID NO: |
| | | | (LIC2003-1: AA SEQ ID NO: | | 41; NA SEQ ID NO: 43) |
| | | | 34; NA SEQ ID NO: 39) | | |
| LIC2004 | 37 | 27 | Forward NKG2D-CD8α-4-1BB- | T2A | IL3-CD8α |
| | | | CD3ζ (LIC2004-1: AA SEQ ID | | (LIC2004-2: AA SEQ ID NO: |
| | | | NO: 35; NA SEQ ID NO: 40) | | 42; NA SEQ ID NO: 44) |

### Extracellular Domain

The chimeric receptors, including the multispecific chimeric receptor, the first chimeric receptor and the second chimeric receptor of the dual chimeric receptor systems, described herein comprise an extracellular domain comprises one or more (such as any one of 1, 2, 3, 4, 5, 6 or more) antigen binding domains, including one or more NKG2D domains and/or a second antigen binding domain. The NKG2D domain and the second antigen binding domain may be fused to each other directly via a peptide bond, via a peptide linker, or via a dimerization motif such as a leucine zipper or cysteine zipper.

### NKG2D Domain

The extracellular domain of the chimeric receptors comprises one or more NKG2D domains. In some embodiments, the extracellular domain of the chimeric receptor comprises a single NKG2D domain. In some embodiments, the extracellular domain comprises a first NKG2D domain and a second NKG2D domain. In some embodiments, the first NKG2D domain and the second NKG2D domain are identical. In some embodiments, the first NKG2D domain and the second NKG2D domain are different. In some embodiments, the first NKG2D domain and the second NKG2D domain are fused to each other directly via a peptide bond or via a peptide linker.

In some embodiments, the NKG2D domain is derived from a human NKG2D molecule. In some embodiments, the NKG2D domain is derived from the extracellular domain of NKG2D, e.g. human NKG2D. In some embodiments, the NKG2D domain is a forward NKG2D domain, i.e., a domain having the amino acid sequence in the same order as the wildtype NKG2D domain. In some embodiments, the NKG2D domain comprises about any one of at least 100, 105, 110, 115, 120, 125, 130, 135, 140, 150 or more amino acids from the extracellular domain of wildtype NKG2D. In some embodiments, the NKG2D domain is a reverse NKG2D domain, i.e. a domain having the reverse sequence as the wildtype NKG2D domain. In some embodiments, the NKG2D domain (including the first NKG2D domain and/or the second NKG2D domain) comprises an amino acid sequence having at least about any one of 85%, 86%, 87%, 88%, 89%, 90%, 91%, 92%, 93%, 94%, 95%, 96%, 97%, 98%, 99%, or 100% sequence identity to SEQ ID NO: 7 or 8. In some embodiments, the NKG2D domain comprises at least 1, 2, 3, 4, 5 or more amino acid substitutions (e.g., conservative amino acid substitutions) compared to the corresponding wildtype sequence of NKG2D.

NKG2D is a unique member of the NKG2 family, which are C-type lectin receptors that stimulate or inhibit cytotoxic activity of NK cells. NKG2D is a type II transmembrane-anchored glycoprotein, expressed primarily on the surface of NK cells and CD8⁺ T cells (e.g., αβ T cells and γδ T cells). It is highly conserved across multiple species, with 70% sequence identity shared between the human and murine receptors. Unlike the other NKG2 receptors that heterodimerize with CD94 and bind to nonclassical MHC glycoproteins class I, NKG2D forms homodimers and bind to cellular stress-inducible molecules. Accumulating evidence indicates that NKG2D plays a crucial role in immunosurveillance against stressed or abnormal cells, such as autologous tumor cells and virus-infected cells.

A variety of NKG2D ligands have been identified in humans, including MIC molecules (MHC class I chain-related proteins A and B, or MICA and MICB) encoded by genes in the MHC family, and ULBP molecules (UL16-binding proteins, also known as RAET1 proteins) which are clustered on human chromosome 6 (Bahram et al. 2005). All NKG2D ligands are homologous to MHC class I molecules and exhibit considerable allelic variation. Although NKG2D ligand RNAs are broadly expressed on all tissues and organs of the body, NKG2D ligands are generally absent from the surface of normal adult cells (Le Bert and Gasser 2014). However, the expression of NKG2D ligands is induced or upregulated primarily in tissues of epithelial origin in response to cellular stress, including heat shock, DNA damage, and stalled DNA replication. Presence of NKG2D ligands on a cell flags the cell for NK cell targeting and potential elimination (Le Bert and Gasser 2014). Interestingly, high activity of DNA repair pathways in transformed cells across a variety of hematologic and solid tumors lead to expression of NKG2D ligands, which renders these cells susceptible to NK-mediated lysis (Sentman et al. 2006).

NKG2D is encoded by KLRK1 gene. NKG2D is a transmembrane receptor protein comprising three domains: cytoplasmic domain (residues 1-51 of human NKG2D), transmembrane domain (residues 52-72 of human NKG2D), and extracellular domain (residues 73-216 of human NKG2D). The extracellular domain of NKG2D contains a C-type lectin domain (residues 98-213 of human NKG2D).

### Second Antigen Binding Domain

The second antigen binding domain specifically binds to a cell surface molecule. The second antigen binding domain may be chosen to recognize an antigen that acts as a cell surface marker on target cells associated with a special disease state. The antigens targeted by the second antigen binding domain may be directly or indirectly involved in the diseases. In some embodiments, the antigen is a tumor antigen. In some embodiments, the tumor antigen is associated with a B cell malignancy.

Tumor antigens are proteins that are produced by tumor cells that can elicit an immune response, particularly T-cell mediated immune responses. The selection of the targeted antigen of the invention will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

The second antigen binding domain can be of any suitable format. In some embodiments, the second antigen binding domain is derived from an antibody, such as a four-chain antibody, or a single-domain antibody, such as heavy-chain only antibody. In some embodiments, the second antigen binding domain is an antibody fragment, such as a Fab, Fv, scFv, or VHH. In some embodiments, the second antigen binding domain is an antibody fragment that specifically binds to an antigen selected from the group consisting of CD19, CD20, CD22, CD33, CD38, BCMA, CS1, ROR1, GPC3, CD123, IL-13R, CD138, c-Met, EGFR, EGFRvIII, GD-2, NY-ESO-1, MAGE A3, and glycolipid F77.

In some embodiments, the second antigen binding domain is a ligand, or a ligand binding domain of a receptor. In some embodiments, the second antigen binding domain is a ligand or ligand binding domain derived from a molecule selected from the group consisting of NKG2A, NKG2C, NKG2F, IL-3, IL-13, LLT1, AICL, DNAM-1, and NKp80.

### CD123-Binding Domain

In some embodiments, the second antigen binding domain is a CD123-binding domain. In some embodiments, the CD123-binding domain is an antibody fragment (e.g., an scFv or a VHH) of an anti-CD123 antibody. In some embodiments, the CD123-binding domain is a ligand of CD123, or an IL-3 domain. In some embodiments, the IL-3 domain is derived from human IL-3, such as full-length or a functional fragment of human IL-3. In some embodiments, the CD123-binding domain comprises an amino acid sequence having at least about any one of 85%, 86%, 87%, 88%, 89%, 90%, 91%, 92%, 93%, 94%, 95%, 96%, 97%, 98%, 99%, or 100% sequence identity to SEQ ID NO: 9.

In some embodiments, there is provided a chimeric receptor comprising: (a) an extracellular domain comprising a CD123-binding domain; and (b) a transmembrane domain. In some embodiments, the transmembrane domain is derived from a molecule selected from the group consisting of CD8α, CD4, CD28, CD137, CD80, CD86, CD152 and PD1. In some embodiments, the chimeric receptor further comprises an intracellular signaling domain. In some embodiments, the intracellular signaling domain comprises a primary intracellular signaling domain of an immune effector cell (such as T cell). In some embodiments, the primary signaling domain is derived from CD3ζ. In some embodiments, the intracellular signaling domain does not comprise a primary intracellular signaling domain of an immune effector cell. In some embodiments, the intracellular signaling domain comprises a co-stimulatory signaling domain. In some embodiments, the intracellular signaling domain consists of (or consists essentially of) one or more co-stimulatory signaling domains. In some embodiments, the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof. In some embodiments, the chimeric receptor further comprises a hinge region (such as a CD8α hinge region) located between the C-terminus of the extracellular domain and the N-terminus of the transmembrane domain. In some embodiments, the chimeric receptor comprises from the N-terminus to the C-terminus: a CD123-binding domain and a transmembrane domain (CD8α). In some embodiments, the CD123-binding domain is an IL-3 domain. In some embodiments, the CD123-binding domain comprises the amino acid sequence of SEQ ID NO: 9.

In some embodiments, there is provided a chimeric receptor comprising an amino acid sequence having at least about any one of 85%, 86%, 87%, 88%, 89%, 90%, 91%, 92%, 93%, 94%, 95%, 96%, 97%, 98%, 99%, or 100% sequence identity to SEQ ID NO: 41 or 42. In some embodiments, there is provided an isolated nucleic acid comprising a nucleic acid sequence having at least about any one of 85%, 86%, 87%, 88%, 89%, 90%, 91%, 92%, 93%, 94%, 95%, 96%, 97%, 98%, 99%, or 100% sequence identity to SEQ ID NO: 43 or 44.

IL-3 (interleukin-3) gene is mapped on chromosome 5, encoding a protein 152 amino acids long. IL-3 is a cytokine, capable of supporting a broad range of cellular activities such as cell growth, differentiation and apoptosis. IL-3 acts by binding to the interleukin-3 receptor (IL-3R), also known as CD123 antigen. IL-3R is a heterodimeric receptor, comprising a ligand specific alpha subunit and a signal transducing beta subunit, shared by the receptors for IL-3, colony stimulating factor 2 (CSF2/GM-CSF), and interleukin 5 (ILS). Activation of the IL-3R results in the phosphorylation of the Pc chain, recruitment of SH2-containing adaptor molecules such as Vav1, and downstream signal transduction via Jak2/STATS and the Ras/MAPK pathway.

IL-3R is a 75 kD glycoprotein and becomes 43 kD when hydrolyzed by N-glycosidase. IL-3R has three extracellular domains which are responsible for specific binding to IL-3, a transmembrane domain, and a short intercellular domain which is indispensable for intracellular signaling (Sato et al. 1993). IL-3R is a heterodimeric receptor with low affinity and high specificity for IL-3. Upon binding to IL-3, the IL-3R is activated and promotes cell proliferation and survival (Liu et al. 2015).

CD123 is overexpressed on AML blasts (i.e., myelobasts). AML blasts and leukemia stem cells (LSCs) in 75 to 89% of AML patients express CD123. In sharp contrast, there is low or undetectable expression of CD123 on normal hematopoietic stem cells (HSCs) (Frankel et al. 2014; Jordan et al. 2000). Apart from AML, CD123 is also overexpressed in a variety of hematologic malignancies, including B cell lineage acute lymphoblastic leukemia, chronic myeloid leukemia, plasmacytoid dendritic cell neoplasm, and hairy cell leukemia (Munoz et al. 2001). This expression profile makes CD123 a valuable biomarker in clinical diagnosis, prognosis and intervention of the diseases. Currently, early phase clinical trials have demonstrated that CD123-targeting therapies are safe and without major adverse effects on hematopoiesis. The anti-leukemic activities of CD123-targeting therapies in humans are still being investigated.

### Dimerization Motif

In some embodiments, the extracellular domain comprises a dimerization motif and a single NKG2D domain. In some embodiments, the extracellular domain comprises the second antigen binding domain, a single NKG2D domain, and a dimerization motif disposed therebetween. The dimerization motif promotes dimerization of two polypeptide chains in a chimeric receptor, thereby facilitating formation of NKG2D homodimer and binding of the NKG2D homodimer to an NKG2D ligand. Suitable dimerization motifs are known in the art. In some embodiments, the dimerization motif is a leucine zipper. In some embodiments, the leucine zipper comprises an amino acid of SEQ ID NO: 10. In some embodiments, the dimerization motif is a cysteine zipper. Exemplary cysteine zippers are known in the art. See, for example, Guilaume et al. (2015) PLoS ONE 10(6): e0128779.

### Peptide Linkers

In some embodiments, the NKG2D domain and the second antigen binding domain (e.g., IL-3 domain) are fused to each other via a peptide linker. In some embodiments, the first NKG2D domain and the second NKG2D domain are fused to each other via a peptide linker. Different domains of the chimeric receptors may also be fused to each other via peptide linkers. The peptide linkers connecting different domains may be the same or different.

Each peptide linker in a chimeric receptor may have the same or different length and/or sequence depending on the structural and/or functional features of the various domains. Each peptide linker may be selected and optimized independently. The length, the degree of flexibility and/or other properties of the peptide linker(s) used in the chimeric receptors may have some influence on properties, including but not limited to the affinity, specificity or avidity for one or more particular antigens or epitopes. For example, longer peptide linkers may be selected to ensure that two adjacent domains do not sterically interfere with one another. In some embodiments, a short peptide linker may be disposed between the transmembrane domain and the intracellular signaling domain of a chimeric receptor. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include glycine polymers (G)ₙ (SEQ ID NO: 28), glycine-serine polymers (including, for example, (GS)ₙ (SEQ ID NO: 29), (GSGGS)ₙ (SEQ ID NO: 30), (GGGS)ₙ (SEQ ID NO: 31), and (GGGGS)ₙ (SEQ ID NO: 32), where n is an integer of at least one), glycine-alanine polymers, alanine-serine polymers, and other flexible linkers known in the art. In some embodiments, the peptide linker comprises an amino acid sequence selected from SEQ ID NOs: 12-15.

### Transmembrane Domain

The chimeric receptors, including the multispecific chimeric receptor, the first chimeric receptor and the second chimeric receptor of the dual chimeric receptor systems, of the present application comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the chimeric receptors described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric receptor described herein is derived from a Type I single-pass membrane protein. In some embodiments, transmembrane domains from multi-pass membrane proteins may also be compatible for use in the chimeric receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the chimeric receptor comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is derived from a molecule selected from the group consisting of CD8α, CD4, CD28, 4-1BB, CD80, CD86, CD152 and PD1.

In some embodiments, the transmembrane domain is derived from CD28. In some embodiments, the transmembrane domain is derived from CD8α. In some embodiments, the transmembrane domain is a full-length transmembrane domain of CD8α. In some embodiments, the transmembrane domain is a truncated transmembrane domain of CD8α. In some embodiments, the transmembrane domain is a transmembrane domain of CD8α comprising the amino acid sequence of SEQ ID NO: 4 or 45.

Transmembrane domains for use in the chimeric receptors described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the chimeric receptor comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

### Intracellular Signaling Domain

The chimeric receptors, including the multispecific chimeric receptor, the first chimeric receptor and the second chimeric receptor of the dual chimeric receptor systems, of the present application comprise an intracellular signaling domain. The intracellular signaling domain is responsible for activation of at least one of the normal effector functions of the immune effector cell expressing the chimeric receptors. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the chimeric receptor comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, FcR gamma (FCER1G), FcR beta (Fc Epsilon Rib), CD3 gamma, CD3 delta, CD3 epsilon, CD5, CD22, CD79a, CD79b, and CD66d.

In some embodiments, the primary intracellular signaling domain is derived from CD3ζ. In some embodiments, the intracellular signaling domain consists of the cytoplasmic signaling domain of CD3ζ. In some embodiments, the primary intracellular signaling domain is a cytoplasmic signaling domain of wildtype CD3ζ. In some embodiments, the primary intracellular signaling domain of wildtype CD3ζ comprises the amino acid sequence of SEQ ID NO: 6.

### Co-Stimulatory Signaling Domain

Many immune effector cells require co-stimulation, in addition to stimulation of an antigen-specific signal, to promote cell proliferation, differentiation and survival, as well as to activate effector functions of the cell. In some embodiments, the intracellular signaling domain comprises at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptors described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains, for example, two copies of the co-stimulatory signaling domain of CD28. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3ζ) and one or more co-stimulatory signaling domains. In some embodiments, the one or more co-stimulatory signaling domains and the primary intracellular signaling domain (such as cytoplasmic signaling domain of CD3ζ) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3ζ). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptors described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACl/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIRL HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

In some embodiments, the one or more co-stimulatory signaling domains are selected from the group consisting of CD27, CD28, 4-1BB (i.e., CD137), ICOS, OX40, CD30, CD40, CD3, lymphocyte function-associated antigen-1 (LFA-1), CD2, CD7, LIGHT, NKG2C, B7-H3 and ligands that specially bind to CD83.

In some embodiments, the intracellular signaling domain in the chimeric receptor of the present application comprises a co-stimulatory signaling domain derived from CD28. In some embodiments, the intracellular signaling domain comprises a cytoplasmic signaling domain of CD3ζ and a co-stimulatory signaling domain of CD28. In some embodiments, the intracellular signaling domain in the chimeric receptor of the present application comprises a co-stimulatory signaling domain derived from 4-1BB (i.e., CD137). In some embodiments, the intracellular signaling domain comprises a cytoplasmic signaling domain of CD3ζ and a co-stimulatory signaling domain of 4-1BB. In some embodiments, the intracellular signaling domain comprises a co-stimulatory signaling domain of 4-1BB comprising the amino acid sequence of SEQ ID NO: 5.

In some embodiments, the intracellular signaling domain in the chimeric receptor of the present application comprises a co-stimulatory signaling domain of CD28 and a co-stimulatory signaling domain of 4-1BB. In some embodiments, the intracellular signaling domain comprises a cytoplasmic signaling domain of CD3, a co-stimulatory signaling domain of CD28, and a co-stimulatory signaling domain of 4-1BB. In some embodiments, the intracellular signaling domain comprises a polypeptide comprising from the N-terminus to the C-terminus: a co-stimulatory signaling domain of CD28, a co-stimulatory signaling domain of 4-1BB, and a cytoplasmic signaling domain of CD3ζ.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Hinge Region

The chimeric receptors, including the multispecific chimeric receptor, the first chimeric receptor and the second chimeric receptor of the dual chimeric receptor systems, of the present application may comprise a hinge region that is located between the extracellular antigen binding domain and the transmembrane domain. A hinge region is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

The hinge region may contain about 10-100 amino acids, e.g., about any one of 15-75 amino acids, 20-50 amino acids, or 30-60 amino acids. In some embodiments, the hinge region may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge region is a hinge region of a naturally occurring protein. Hinge regions of any protein known in the art to comprise a hinge region are compatible for use in the chimeric receptors described herein. In some embodiments, the hinge region is at least a portion of a hinge region of a naturally occurring protein and confers flexibility to the chimeric receptor. In some embodiments, the hinge region is derived from CD8α. In some embodiments, the hinge region is a portion of the hinge region of CD8α, e.g., a fragment containing at least 15 (e.g., 20, 25, 30, 35, or 40) consecutive amino acids of the hinge region of CD8α. In some embodiments, the hinge region of CD8α comprises the amino acid sequence of SEQ ID NO: 3.

Hinge regions of antibodies, such as an IgG, IgA, IgM, IgE, or IgD antibodies, are also compatible for use in the chimeric receptors described herein. In some embodiments, the hinge region is the hinge region that joins the constant domains CH1 and CH2 of an antibody. In some embodiments, the hinge region is of an antibody and comprises the hinge region of the antibody and one or more constant regions of the antibody. In some embodiments, the hinge region comprises the hinge region of an antibody and the CH3 constant region of the antibody. In some embodiments, the hinge region comprises the hinge region of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge regions for the chimeric receptors described herein. In some embodiments, the hinge region between the C-terminus of the extracellular ligand-binding domain of an Fc receptor and the N-terminus of the transmembrane domain is a peptide linker, such as a (G×S)n linker, wherein x and n, independently can be an integer between 3 and 12, including 3, 4, 5, 6, 7, 8, 9, 10, 11, 12, or more.

### Signal Peptide

The chimeric receptors, including the multispecific chimeric receptor, the first chimeric receptor and the second chimeric receptor of the dual chimeric receptor systems, of the present application may comprise a signal peptide (also known as a signal sequence) at the N-terminus of the polypeptide(s). In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the chimeric receptors described herein will be evident to one of skill in the art. In some embodiments, the signal peptide is derived from a molecule selected from the group consisting of CD8α, GM-CSF receptor α, IL-3, and IgG1 heavy chain. In some embodiments, the signal peptide is derived from CD8α. In some embodiments, the signal peptide of IL-3 comprises the amino acid sequence of SEQ ID NO: 1. In some embodiments, the signal peptide of CD8α comprises the amino acid sequence of SEQ ID NO: 2.

### III. Engineered Immune Effector Cells

Further provided in the present application are host cells (such as engineered immune effector cells) comprising any one of the chimeric receptors, multispecific chimeric receptors or dual chimeric receptor systems described herein.

Thus, in some embodiments, there is provided an engineered immune effector cell (such as T cell) comprising a chimeric receptor comprising: (a) an extracellular domain comprising an NKG2D domain; (b) a transmembrane domain; and (c) an intracellular signaling domain. In some embodiments, the extracellular domain comprises a first NKG2D domain and a second NKG2D domain.

In some embodiments, there is provided an engineered immune effector cell (such as T cell) comprising a multispecific (e.g., bispecific) chimeric receptor comprising: (a) an extracellular domain comprising an NKG2D domain and a second antigen binding domain (e.g., an IL-3 domain); (b) a transmembrane domain; and (c) an intracellular signaling domain.

In some embodiments, there is provided an engineered immune effector cell (such as T cell) comprising a multispecific (e.g., bispecific) chimeric receptor comprising a polypeptide chain comprising: (a) an extracellular domain comprising a first NKG2D domain, a second NKG2D domain, and a CD123 binding domain (e.g., an IL-3 domain); (b) a transmembrane domain; and (c) an intracellular signaling domain. In some embodiments, the CD123 binding domain is an anti-CD123 antibody fragment (e.g., scFv or VHH). In some embodiments, the CD123 binding domain is an IL-3 domain. In some embodiments, the polypeptide chain comprises from the N-terminus to the C-terminus: an IL-3 domain, a first peptide linker, the first NKG2D domain, a second peptide linker, the second NKG2D domain, a CD8α hinge region, a CD8α transmembrane domain, a co-stimulatory signaling domain derived from 4-1BB, and a primary intracellular signaling domain derived from CD3ζ.

In some embodiments, there is provided an engineered immune effector cell (such as T cell) comprising a multispecific (e.g., bispecific) chimeric receptor comprising a first polypeptide chain and a second polypeptide chain each comprising: (a) an extracellular domain comprising an NKG2D domain and a CD123 binding domain (e.g., an IL-3 domain); (b) a transmembrane domain; and (c) an intracellular signaling domain. In some embodiments, the NKG2D domain of the first polypeptide chain is crosslinked to the NKG2D domain of the second polypeptide chain via one or more disulfide bonds. In some embodiments, each of the first polypeptide chain and the second polypeptide chain comprises from the N-terminus to the C-terminus: an IL-3 domain, a peptide linker, the NKG2D domain, a CD8α hinge region, a CD8α transmembrane domain, a co-stimulatory signaling domain derived from 4-1BB, and a primary intracellular signaling domain derived from CD3ζ. In some embodiments, the extracellular domain further comprises a dimerization motif (e.g., a leucine zipper or cysteine zipper) disposed between the NKG2D domain and the CD123 binding domain. In some embodiments, each of the first polypeptide chain and the second polypeptide chain comprises from the N-terminus to the C-terminus: an IL-3 domain, a leucine zipper, the NKG2D domain, a CD8α hinge region, a CD8α transmembrane domain, a co-stimulatory signaling domain derived from 4-1BB, and a primary intracellular signaling domain derived from CD3ζ.

In some embodiments, there is provided an engineered immune effector cell (such as T cell) comprising a dual chimeric receptor system comprising: (i) a first chimeric receptor comprising a first polypeptide chain and a second polypeptide chain each comprising: (a) a first extracellular domain comprising an NKG2D domain; (b) a first transmembrane domain; and (c) a first intracellular signaling domain; and (ii) a second chimeric receptor comprising a third polypeptide chain comprising: (a) a second extracellular domain comprising a second antigen binding domain; (b) a second transmembrane domain; and optionally (c) a second intracellular signaling domain. In some embodiments, each of the first polypeptide chain and the second polypeptide chain comprises from the N-terminus to the C-terminus: the NKG2D domain, a CD8α hinge region, a CD8α transmembrane domain, a co-stimulatory signaling domain derived from 4-1BB, and a primary intracellular signaling domain derived from CD3ζ. In some embodiments, the second chimeric receptor comprises a polypeptide comprising from the N-terminus to the C-terminus: an IL-3 domain, a CD8α hinge region, a CD8α transmembrane domain, and optionally a co-stimulatory signaling domain derived from 4-1BB.

In some embodiments, there is provided an engineered immune effector cell (such as T cell) comprising a dual chimeric receptor system comprising: (i) a first chimeric receptor comprising a first polypeptide chain comprising: (a) a first extracellular domain comprising a first NKG2D domain and a second NKG2D domain; (b) a first transmembrane domain; and (c) a first intracellular signaling domain; and (ii) a second chimeric receptor comprising a second polypeptide chain comprising: (a) a second extracellular domain comprising a second antigen binding domain; (b) a second transmembrane domain; and optionally (c) a second intracellular signaling domain. In some embodiments, the first chimeric receptor comprises a polypeptide comprising from the N-terminus to the C-terminus: a first NKG2D domain, a second NKG2D domain, a CD8α hinge region, a CD8α transmembrane domain, a co-stimulatory signaling domain derived from 4-1BB, and a primary intracellular signaling domain derived from CD3.

In some embodiments, the second chimeric receptor comprises a polypeptide comprising from the N-terminus to the C-terminus: an IL-3 domain, a CD8α hinge region, a CD8α transmembrane domain, and optionally a co-stimulatory signaling domain derived from 4-1BB.

In some embodiments, the engineered immune effector cell expresses one or more immunomodulators. In some embodiments, the engineered mammalian cell comprises a heterologous nucleic acid encoding the immunomodulator. In some embodiments, the heterologous nucleic acid encoding the immunomodulatory is present on the vector encoding the chimeric receptor, multispecific chimeric receptor or the dual chimeric receptor system described herein. In some embodiments, the immunomodulator is a regulator of Runx3. In some embodiments, the regulator is a polypeptide, such as a polypeptide derived from Runx3 or MITF. In some embodiments, the regulator is an RNA, such as miRNA or siRNA. Runx3 may help T cell homing and infiltrating into solid tumor, which is an important factor in a successful T-cell mediated immunotherapy.

### Vectors

The present application provides vectors for cloning and expressing any one of the chimeric receptors, multispecific chimeric receptors or dual chimeric receptor systems described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying chimeric receptors, multispecific chimeric receptors or dual chimeric receptor systems can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a chimeric receptor, multispecific chimeric receptor or dual chimeric receptor system described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present invention. Promoters may be roughly categorized as constitutive promoters or regulated promoters, such as inducible promoters.

In some embodiments, the nucleic acid encoding the chimeric receptor, multispecific chimeric receptor or dual chimeric receptor system is operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken (3-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)).

In some embodiments, the nucleic acid encoding the chimeric receptor, multispecific chimeric receptor or dual chimeric receptor system is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof. In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the chimeric receptor, multispecific chimeric receptor or dual chimeric receptor system from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### Immune Effector Cells

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the chimeric receptor, multispecific chimeric receptor, or dual chimeric receptor system and binding to the target cells, such as CD20+ or CD19+ tumor cells. In some embodiments, the CD8+ T cells lyse antigen-specific target cells upon expressing the chimeric receptor, multispecific chimeric receptor or dual chimeric receptor system and binding to the target cells.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

The engineered immune effector cells are prepared by introducing the chimeric receptors, multispecific chimeric receptors or dual chimeric receptor systems into the immune effector cells, such as T cells. In some embodiments, the chimeric receptor, multispecific chimeric receptor or dual chimeric receptor system is introduced to the immune effector cells by transfecting any one of the isolated nucleic acids or any one of the vectors described herein. In some embodiments, the chimeric receptor, multispecific chimeric receptor or dual chimeric receptor system is introduced to the immune effector cells by inserting proteins into the cell membrane while passing cells through a microfluidic system, such as CELL SQUEEZE® (see, for example, U.S. Patent Application Publication No. 20140287509).

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the chimeric receptors, multispecific chimeric receptors or dual chimeric receptor systems described herein may be prepared by a conventional method (e.g., in vitro transcription) and then introduced into the immune effector cells via known methods such as mRNA electroporation. See, e.g., Rabinovich et al., Human Gene Therapy 17: 1027-1035.

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding the chimeric receptors, multispecific chimeric receptors or dual chimeric receptor systems in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### Sources of T Cells

Prior to expansion and genetic modification of the T cells, a source of T cells is obtained from an individual. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present application is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells with the chimeric receptors, multispecific chimeric receptors or dual chimeric receptor systems described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### IV. Pharmaceutical Compositions

Further provided by the present application are pharmaceutical compositions comprising any one of the engineered immune effector cells comprising any one of the chimeric receptors, multispecific chimeric receptors or dual chimeric receptor systems as described herein, and a pharmaceutically acceptable carrier. Pharmaceutical compositions can be prepared by mixing a plurality of engineered immune effector cells having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1 to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/mL to about 1.0 mg/mL, preferably about 0.07 mg/mL to about 0.2 mg/mL.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The pharmaceutical compositions described herein may also contain more than one active compound or agent as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

### V. Methods of Treating Cancer

The present application further relates to methods and compositions for use in cell immunotherapy. In some embodiments, the cell immunotherapy is for treating cancer, including but not limited to hematological malignancies and solid tumors. Any of the chimeric receptors, multispecific chimeric receptors, dual chimeric receptor systems, and engineered immune effector cells (such as engineered T cells) described herein may be used in the method of treating cancer.

In some embodiments, there is provided a method of treating a cancer (such as multiple myeloma, acute lymphoblastic leukemia, or chronic lymphocytic leukemia) in an individual (such as a human individual), comprising administering to the individual an effective amount of a pharmaceutical composition comprising: (1) an engineered immune effector cell (such as T cell) comprising a chimeric receptor comprising: (a) an extracellular domain comprising an NKG2D domain; (b) a transmembrane domain; and (c) an intracellular signaling domain; and (2) a pharmaceutically acceptable carrier. In some embodiments, the chimeric receptor comprises a polypeptide comprising from the N-terminus to the C-terminus: a first NKG2D domain, a second NKG2D domain, a CD8α hinge region, a CD8α transmembrane domain, a co-stimulatory signaling domain derived from 4-1BB, and a primary intracellular signaling domain derived from CD3.

In some embodiments, there is provided a method of treating a cancer (such as multiple myeloma, acute lymphoblastic leukemia, or chronic lymphocytic leukemia) in an individual (such as a human individual), comprising administering to the individual an effective amount of a pharmaceutical composition comprising: (1) an engineered immune effector cell (such as T cell) comprising a multispecific chimeric receptor comprising a polypeptide chain comprising: (a) an extracellular domain comprising a first NKG2D domain, a second NKG2D domain, and a CD123 binding domain (e.g., an IL-3 domain); (b) a transmembrane domain; and (c) an intracellular signaling domain; and (2) a pharmaceutically acceptable carrier. In some embodiments, the CD123 binding domain is an anti-CD123 antibody fragment (e.g., scFv or VHH). In some embodiments, the CD123 binding domain is an IL-3 domain. In some embodiments, the polypeptide chain comprises from the N-terminus to the C-terminus: an IL-3 domain, a first peptide linker, the first NKG2D domain, a second peptide linker, the second NKG2D domain, a CD8α hinge region, a CD8α transmembrane domain, a co-stimulatory signaling domain derived from 4-1BB, and a primary intracellular signaling domain derived from CD3.

In some embodiments, there is provided a method of treating a cancer (such as multiple myeloma, acute lymphoblastic leukemia, or chronic lymphocytic leukemia) in an individual (such as a human individual), comprising administering to the individual an effective amount of a pharmaceutical composition comprising: (1) an engineered immune effector cell (such as T cell) comprising a multispecific chimeric receptor comprising a first polypeptide chain and a second polypeptide chain each comprising: (a) an extracellular domain comprising an NKG2D domain and a CD123 binding domain (e.g., an IL-3 domain); (b) a transmembrane domain; and (c) an intracellular signaling domain; and (2) a pharmaceutically acceptable carrier. In some embodiments, the NKG2D domain of the first polypeptide chain is crosslinked to the NKG2D domain of the second polypeptide chain via one or more disulfide bonds. In some embodiments, each of the first polypeptide chain and the second polypeptide chain comprises from the N-terminus to the C-terminus: an IL-3 domain, a peptide linker, the NKG2D domain, a CD8α hinge region, a CD8α transmembrane domain, a co-stimulatory signaling domain derived from 4-1BB, and a primary intracellular signaling domain derived from CD3ζ. In some embodiments, the extracellular domain further comprises a dimerization motif (e.g., a leucine zipper or cysteine zipper) disposed between the NKG2D domain and the CD123 binding domain. In some embodiments, each of the first polypeptide chain and the second polypeptide chain comprises from the N-terminus to the C-terminus: an IL-3 domain, a leucine zipper, the NKG2D domain, a CD8α hinge region, a CD8α transmembrane domain, a co-stimulatory signaling domain derived from 4-1BB, and a primary intracellular signaling domain derived from CD3.

In some embodiments, there is provided a method of treating a cancer (such as multiple myeloma, acute lymphoblastic leukemia, or chronic lymphocytic leukemia) in an individual (such as a human individual), comprising administering to the individual an effective amount of a pharmaceutical composition comprising: (1) an engineered immune effector cell (such as T cell) comprising a dual chimeric receptor system comprising: (i) a first chimeric receptor comprising a first polypeptide chain and a second polypeptide chain each comprising: (a) a first extracellular domain comprising an NKG2D domain; (b) a first transmembrane domain; and (c) a first intracellular signaling domain; and (ii) a second chimeric receptor comprising a third polypeptide chain comprising: (a) a second extracellular domain comprising a second antigen binding domain; (b) a second transmembrane domain; and optionally (c) a second intracellular signaling domain; and (2) a pharmaceutically acceptable carrier. In some embodiments, each of the first polypeptide chain and the second polypeptide chain comprises from the N-terminus to the C-terminus: the NKG2D domain, a CD8α hinge region, a CD8α transmembrane domain, a co-stimulatory signaling domain derived from 4-1BB, and a primary intracellular signaling domain derived from CD3ζ. In some embodiments, the second chimeric receptor comprises a polypeptide comprising from the N-terminus to the C-terminus: an IL-3 domain, a CD8α hinge region, a CD8c transmembrane domain, and optionally a co-stimulatory signaling domain derived from 4-1BB.

The methods described herein are suitable for treating various cancers, including both solid cancer and liquid cancer. In some embodiments, the cancer is multiple myeloma, acute lymphoblastic leukemia, or chronic lymphocytic leukemia. In some embodiments, the cancer is refractory or relapsed cancer. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

Administration of the pharmaceutical compositions may be carried out in any convenient manner, such as injection, transfusion, implantation or transplantation. The compositions may be administered to a patient transarterially, subcutaneously, intradermally, intratumorally, intranodally, intramedullary, intramuscularly, intravenously, or intraperitoneally. In some embodiments, the pharmaceutical composition is administered systemically. In some embodiments, the pharmaceutical composition is administered to an individual by infusion, such as intravenous infusion. Infusion techniques for immunotherapy are known in the art (see, e.g., Rosenberg et al., New Eng. J. of Med. 319: 1676 (1988)). In some embodiments, the compositions are administered by intravenous injection.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

In some embodiments, the amount of the pharmaceutical composition is effective to cause an objective clinical response in the individual. In some embodiments, the amount of the pharmaceutical composition is effective to cause disease remission (partial or complete) in the individual. In some embodiments, the amount of the pharmaceutical composition is effective to prevent relapse or disease progression of the cancer in the individual. In some embodiments, the amount of the pharmaceutical composition is effective to prolong survival (such as disease free survival) in the individual. In some embodiments, the pharmaceutical composition is effective to improve quality of life in the individual.

In some embodiments, the amount of the pharmaceutical composition is effective to inhibit growth or reducing the size of a solid or lymphatic tumor. In some embodiments, the size of the solid or lymphatic tumor is reduced for at least about 10% (including for example at least about any of 20%, 30%, 40%, 60%, 70%, 80%, 90%, or 100%).

In some embodiments, the amount of the pharmaceutical composition is effective to inhibit tumor metastasis in the individual. In some embodiments, at least about 10% (including for example at least about any of 20%, 30%, 40%, 60%, 70%, 80%, 90%, or 100%) metastasis is inhibited. In some embodiments, a method of inhibiting metastasis to lymph node is provided. In some embodiments, a method of inhibiting metastasis to the lung is provided. Metastasis can be assessed by any known methods in the art, such as by blood tests, bone scans, x-ray scans, CT scans, PET scans, and biopsy.

### VI. Kits and Articles of Manufacture

Further provided are kits, unit dosages, and articles of manufacture comprising any of the chimeric receptors, multispecific chimeric receptors, dual chimeric receptor systems, or the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the present application and should therefore not be considered to limit the invention in any way. The following examples and detailed description are offered by way of illustration and not by way of limitation.

### Example 1. Preparation of NKG2D×IL-3 Chimeric Receptors and Dual NKG2D/IL-3 Chimeric Receptor Systems

This example describes design and preparation of exemplary bispecific chimeric receptors, dual chimeric receptor systems and engineered T cells.

### Design of Bispecific Chimeric Receptor and Dual Chimeric Receptor System Constructs

Tables 1 and 2 show the components of the seven constructs and corresponding sequences.

Construct 1: monomeric NKG2D×IL-3 chimeric receptor (LIC2001). This bispecific chimeric receptor comprises a single polypeptide chain comprising from the N-terminus to the C-terminus an extracellular domain, a transmembrane domain, and an intracellular signaling domain, wherein the extracellular domain comprises from the N-terminus to the C-terminus an IL-3 domain specific for CD123 binding, a first peptide linker, a first NKG2D domain having an arginine residue engineered at its N-terminus, a second peptide linker, and a second NKG2D domain having an aspartic acid residue engineered at its C-terminus. The NKG2D domains are responsible for NKG2D ligand binding upon dimerization. The engineered arginine and aspartic acid residues can associate with each other to prevent binding of the NKG2D dimer to free NKG2D ligands that are not bound to tumor cells. The nucleic acid construct encodes the single polypeptide chain.

Construct 2: monomeric NKG2D×IL-3 chimeric receptor (LIC2001-1). This bispecific chimeric receptor comprises a single polypeptide chain comprising from the N-terminus to the C-terminus an extracellular domain, a transmembrane domain, and an intracellular signaling domain, wherein the extracellular domain comprises from the N-terminus to the C-terminus an IL-3 domain specific for CD123 binding, a first peptide linker, a first NKG2D domain, a second peptide linker, and a second NKG2D domain. The NKG2D domains are responsible for NKG2D ligand binding upon dimerization. The nucleic acid construct encodes the single polypeptide chain. For comparison, a monospecific NKG2D chimeric receptor (LIC2001-2) was constructed, which comprises from the N-terminus to the C-terminus an extracellular domain comprising a first forward NKG2D domain, a peptide linker, and a second forward NKG2D domain; a transmembrane domain (CD8α), and an intracellular signaling domain (4-1BB and CD3ζ). The amino acid sequence of LIC2001-2 is SEQ ID NO: 33, and the nucleic acid sequence of LIC2001-2 is SEQ ID NO: 38.

Construct 3: dimeric NKG2D×IL-3 chimeric receptor with a leucine zipper motif (LIC2002). This bispecific chimeric receptor comprises two identical polypeptide chains each comprising from the N-terminus to the C-terminus an extracellular domain, a transmembrane domain, and an intracellular signaling domain, wherein the extracellular domain comprises from the N-terminus to the C-terminus an IL-3 domain specific for CD123 binding, a leucine zipper motif, and a reverse NKG2D domain responsible for NKG2D ligand binding upon homodimerization. The nucleic acid construct encodes a single copy of the polypeptide chain.

Construct 4: dimeric NKG2D×IL-3 chimeric receptor without a leucine zipper motif (LIC2002-1). This bispecific chimeric receptor comprises two identical polypeptide chains each comprising from the N-terminus to the C-terminus an extracellular domain, a transmembrane domain, and an intracellular signaling domain, wherein the extracellular domain comprises from the N-terminus to the C-terminus an IL-3 domain specific for CD123 binding, a peptide linker, and a reverse NKG2D domain responsible for NKG2D ligand binding upon homodimerization. The nucleic acid construct encodes a single copy of the polypeptide chain.

Construct 5: dimeric NKG2D×IL-3 chimeric receptor with a leucine zipper motif (LIC2002-2). This bispecific chimeric receptor comprises two identical polypeptide chains each comprising from the N-terminus to the C-terminus an extracellular domain, a transmembrane domain, and an intracellular signaling domain, wherein the extracellular domain comprises from the N-terminus to the C-terminus an IL-3 domain specific for CD123 binding, a leucine zipper motif, and a forward NKG2D domain responsible for NKG2D ligand binding upon homodimerization. The nucleic acid construct encodes a single copy of the polypeptide chain.

Construct 6: dual NKG2D/IL-3 chimeric receptor system (LIC2003). A polycistronic nucleic acid construct for a dual NKG2D/IL-3 chimeric receptor system is designed. The construct encodes for a first polypeptide comprising from the N-terminus to the C-terminus: an extracellular domain comprising a reverse NKG2D domain and a forward NKG2D domain, responsible for NKG2D ligand binding upon homodimerization, a transmembrane domain and an intracellular signaling domain, followed by T2A self-cleaving peptide, and a second polypeptide comprising from the N-terminus to the C-terminus: an IL-3 domain specific for CD123 binding, followed by a transmembrane domain without an intracellular signaling domain. Upon expression of the construct, the first polypeptide forms the first chimeric receptor, and the second polypeptide forms the second chimeric receptor.

Construct 7: dual NKG2D/IL-3 chimeric receptor system (LIC2004). A polycistronic nucleic acid construct for a dual NKG2D/IL-3 chimeric receptor system is designed. The construct encodes for a first polypeptide comprising from the N-terminus to the C-terminus: an extracellular domain comprising a forward NKG2D domain, responsible for NKG2D ligand binding upon homodimerization, a transmembrane domain and an intracellular signaling domain, followed by T2A self-cleaving peptide, and a second polypeptide comprising from the N-terminus to the C-terminus: an IL-3 domain specific for CD123 binding, followed by a transmembrane domain without an intracellular signaling domain. Upon expression of the construct, the two copies of the first polypeptide form the first chimeric receptor, which is dimeric, and the second polypeptide forms the second chimeric receptor. A monospecific NKG2D chimeric receptor (LIC2004-1) was constructed, which comprises from the N-terminus to the C-terminus an extracellular domain comprising a forward NKG2D domain, a CD8α hinge domain; a transmembrane domain (CD8α), and an intracellular signaling domain (4-1BB and CD3). The amino acid sequence of LIC2004-1 is SEQ ID NO: 35, and the nucleic acid sequence of LIC2004-1 is SEQ ID NO: 40.

### Generation of Lentivirus Expression Vectors

Briefly, a lentiviral vector was modified using pLVX-Puro (Clontech #632164) by replacing the original promoter with human elongation factor 1a promoter (hEF1α) gene with EcoRI and XbaI by GenScript. NKG2D×IL-3 chimeric receptor genes or NKG2D/IL-3 dual chimeric receptor system genes were constructed by GenScript, and cloned into the vector via EcoRI/HpaI to provide recombination lentivirus expression plasmid, which was further subject to the lentivirus packaging procedure.

The lentivirus packaging plasmid mixture including pMDLg/pRRE (Addgene #12251), pRSV-Rev (Addgene #12253), and pMD2.G (Addgene #12259) was pre-mixed with an pLVX-NKG2D×IL-3 chimeric receptor/dual chimeric receptor system-Puro expression plasmid at a pre-optimized ratio with polyetherimide (PEI), then mixed properly and incubated at room temperature for 5 minutes. The transfection mix was then added dropwise to the 293FT cells and mixed gently. Afterwards, cells were incubated overnight in a 37° C. and 5% CO₂ cell incubator. The supernatants were collected after centrifugation at 4° C., 500 g for 10 min. After the supernatants were filtered through a 0.45 μM PES filter, the virus supernatants were concentrated with 20% sucrose gradient ultracentrifugation. After centrifugation, the supernatants were carefully discarded and the virus pellets were rinsed cautiously with pre-chilled DPBS. The concentration of virus was then measured. Virus was aliquoted properly, then stored at −80° C. immediately. The virus titer was determined by p24 based on HTRF kit developed by GenScript. The following recombinant lentivirus expression plasmids were prepared corresponding to each of the seven constructs described above: pLLV-LIC2001, pLLV-LIC2001-1, pLLV-LIC2002, pLLV-LIC2002-1, pLLV-LIC2002-2, pLLV-LIC2003, and pLLV-LIC2004, as well as pLLV-LIC2001-2 and pLLV-LIC2004-1.

### PBMC Preparation

Leukocytes were collected, and cell concentration was adjusted to 5×10⁶ cells/mL in R10 medium. Leukocytes were then mixed with 0.9% NaCl solution at 1:1 (v/v) ratio. 3 mL lymphoprep medium was added to a 15 mL centrifuge tube, and on top of lymphoprep was slowly layered 6 mL of diluted lymphocyte mix. The lymphocyte mix was centrifuged at 800 g for 30 minutes without brakes at 20° C. Lymphocyte buffy coat was then collected with a 200 μL pipette. The harvested fraction was diluted with at least 6 folds of 0.9% NaCl or R10 to reduce the density of the solution. The harvested fraction was then centrifuged at 250 g for 10 minutes at 20° C. The supernatant was aspirated completely, and 10 mL of R10 was added to the cell pellet. The mixture was further centrifuged at 250 g for 10 minutes at 20° C. The supernatant was then aspirated. 2 mL 37° C. pre-warmed R10 with 100 IU/mL IL-2 was added to the cell pellet, and the cell pellet was resuspended softly. The number of cells was then counted, and the PBMC sample was ready for later experiments.

### T Cell Purification

Human T cells were purified from PBMCs using Miltenyi Pan T cell isolation kit (Cat #130-096-535), following the protocol provided by the manufacturer as below. Cell number was first determined. The cell suspension was centrifuged at 300 g for 10 minutes. Supernatant was then aspirated completely, and cell pellets were resuspended in 40 μL buffer per 10⁷ total cells. 10 μL of Pan T Cell Biotin-Antibody Cocktail was added per 10⁷ total cells, mixed thoroughly and incubated for about 5 minutes in the refrigerator (2˜8° C.). 30 μL of buffer was then added per 10⁷ cells. 20 μL of Pan T Cell MicroBead Cocktail was added per 10⁷ cells. The mixture was mixed well and incubated for an additional 10 minutes in the refrigerator (2˜8° C.). A minimum of 500 μL was required for magnetic separation. LS column was placed in the magnetic field of a suitable MACS Separator. The column was prepared by rinsing with 3 mL of buffer. Cell suspension was then applied onto the column, and flow-through containing unlabeled cells was collected, representing the enriched T cell fractions. T cells were then collected by washing column with 3 mL of buffer, collecting unlabeled cells that pass through, which represent the enriched T cells, and combining with the flow-through from previous step. T cells were then resuspended in R10+100 IU/mL IL-2. The primary T cells were then pre-activated with human T Cell Activation/Expansion Kit (Miltenyi #130-091-441) for 3 days prior to transduction.

Purified T cells were transfected with each of the recombinant lentiviral vectors, or transfected with mRNA encoding each of the chimeric receptor constructs through electroporation, then incubated overnight at 37° C., 5% CO₂ incubator. Engineered T cells expressing each of the seven constructs described in this Example were prepared.

### Expression of Chimeric Receptors on Engineered T Cells

mRNA molecules encoding the LIC2002-2 and LIC2004 chimeric receptor constructs were delivered respectively to T cells by electroporation. Expression of the chimeric receptors was detected using a flow cytometry assay. Briefly, electroporated T cells were harvested and rinsed with DPBS, and then resuspended in 100 μL DPBS containing 2 μL PE-conjugated CD314 protein (MILTENYI BIOTEC, 130-111-645) for NKG2D domain detection, or 10 μL PE-conjugated anti-IL-3 antibody (MILTENYI BIOTEC, 130-096-084) for IL-3 domain detection. The reaction mixtures were incubated for 20 min at 4° C. Subsequently, cells were washed with 200 μL DPBS, resuspended in DPBS, and analyzed by flow cytometry. As shown in FIG. 2, the engineered T cells expressed the chimeric receptor(s) having both NKG2D and IL-3 domains.

### In Vitro Cytotoxicity Assay

The engineered T cells were harvested and inoculated in a 384-well reaction plate. Target cells were human chronic myelogenous leukemia (CIVIL) cell lines K562-Luc and K562-CD123-Luc, which recombinantly expressed CD123. All of the cell lines were engineered in-house to express firefly luciferase. To assay the cytotoxicity of the engineered T cells against tumor cells, the engineered T cells were co-incubated with the target cells at an effector (engineered T cells) to target cell ratio (“E:T ratio”) of 20:1 for 20 hours. ONE-GLO™ luminescent luciferase assay reagents (Promega #E6110) were prepared according to manufacturer's protocol and added to the co-cultured cells to detect the remaining luciferase activity in each well. Since luciferase was expressed only in the target cells, the remaining luciferase activity in a well correlated directly to the number of viable target cells in the well. The maximum luciferase activity was obtained by adding culture media to target cells in the absence of effector cells. The minimum luciferase activity was determined by adding Triton X-100 at a final concentration of 1% as positive control. Specific Lysis/Cytotoxicity was calculated according to the formula:

Specific Lysis/Cytotoxicity %=100%×[1−(LUCsample−LUCmin)/(LUCmax−LUCmin)]

The luciferase value (“LUC” or Luminescence) is proportional to the amount of viable cells in each reaction well.

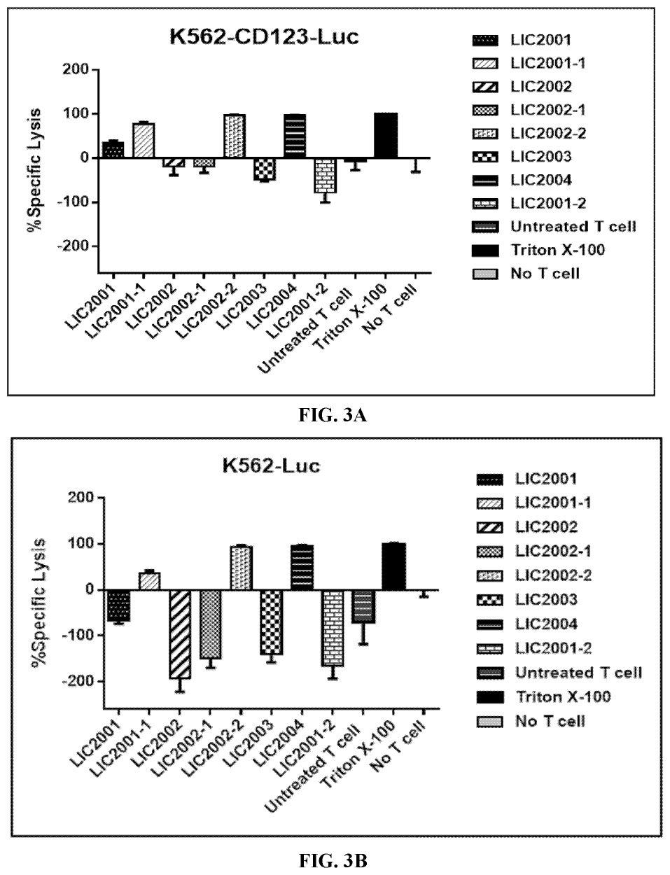

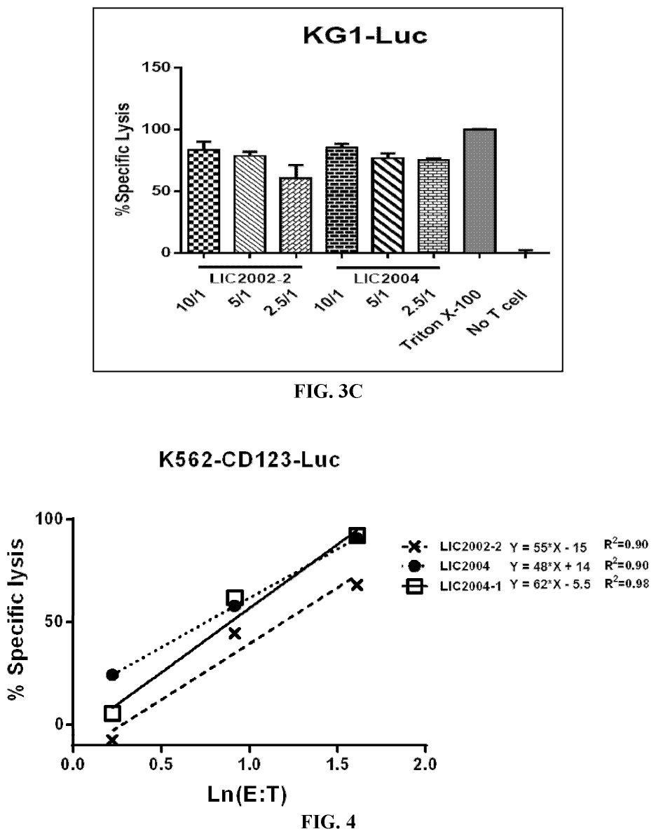

As shown in FIG. 3A, LIC2001-1 expressing T cells (76.63±4.58%), LIC2002-2 expressing T cells (96.52±1.68%) and LIC2004 expressing T cells cell (96.89±0.70%) demonstrated significantly killing effect against K562-CD123-Luc. As shown in FIG. 3B, killing effects were also observed for K562-Luc cells: LIC2001-1 expressing T cells (35.98±6.08%), LIC2002-2 expressing T cells (94.42±2.66%), LIC2004 expressing T cells (95.87±1.61%). However, the engineered T cells expressing the various NKG2D×IL-3 chimeric receptor constructs showed higher cytotoxicity against K562-CD123-Luc cells than K562-Luc cells.

LIC2002-2 expressing T cells and LIC2004 expressing T cells were co-incubated with human acute myelogenous leukemia (AML) cell line KG1-Luc at the effector (engineered T cells) to target cell ratio of 10:1, 5:1 or 2.5:1 to assess in vitro cytotoxicity against KG1-Luc. As shown in FIG. 3C, potent and dose-dependent cytotoxicity effects were observed for both LIC2002-2 expressing T cells (83.68±7%, 78.55±4%, and 60.87±10%) and LIC2004 expressing T cells (85.6±3%, 76.89±4%, and 75.53±1%).

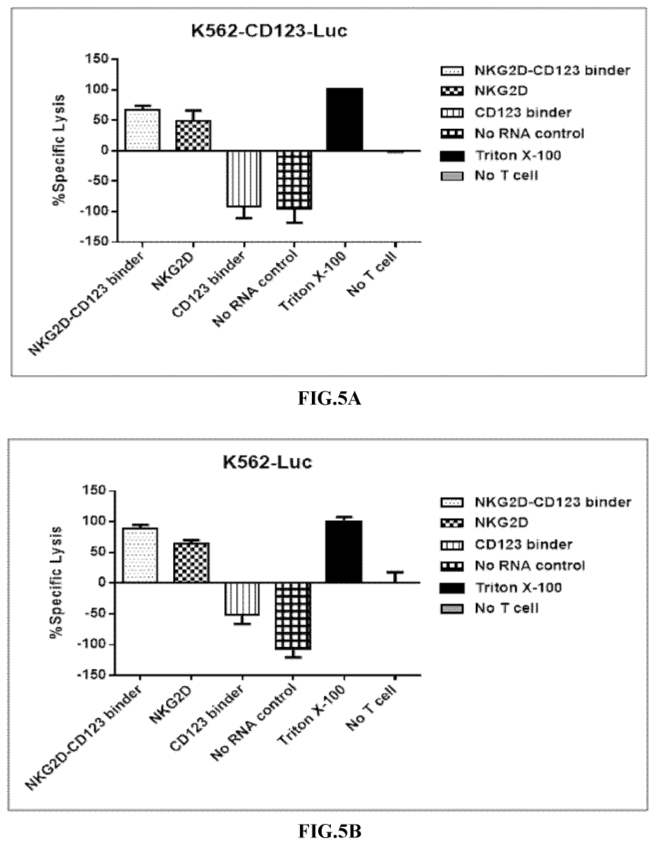

The cytotoxic activities of LIC2002-2 expressing T, LIC2004 expressing T and LIC2004-1 expressing T against K562-CD123-Luc at various effector:target ratios were compared. The LIC2004-1 construct is the NKG2D chimeric receptor in the LIC2004 dual chimeric receptor system. The results are shown in FIG. 4. The Y-axis shows the percentage of specific killing, and the X-axis shows natural logarithm of the effector:target ratio (i.e., engineered T cells: K562-CD123-Luc cells). The logarithm of the E:T ratio is plotted against the percentage of specific killing, which is fitted to the dotted line by linear regression. A smaller slope of the fitted line presents a stronger killing ability. The results demonstrate that the LIC2004 dual chimeric receptor system has higher efficacy than the corresponding NKG2D chimeric receptor alone (i.e., LIC2004-1; p=0.031). There is no significant difference between the efficacy of the bispecific chimeric receptor construct LIC2002-2 and LIC2004 (p=0.277). Statistical analysis was performed using Graphpad Prism 6.

### Mechanism of Action

A series of in vitro cell cytotoxicity assays were performed to study the mechanism of the engineered T cells expressing NKG2D×IL-3 chimeric receptor constructs. Firstly, “NKG2D-CD123 T cells” (LIC2004 expressing T cells), “NKG2D T cells” (T cells expressing NKG2D-CD8hinge-CD8TM-4-1BB-CD3ζ), and “CD123 binder T cells” (T cells expressing IL3-CD8hinge-CD8TM) were co-cultured with target K562-CD123-luc and K562-luc cells respectively at an E:T ratio of 20:1.

As shown in FIG. 5A, NKG2D-CD123 T cells (70.59±1.5%) showed significant tumor killing effect, while the tumor killing effect of NKG2D T cells (42.14±8.4%) was much weaker, and CD123 binder T cells showed no cytotoxicity against the target tumor cells. In FIG. 5B, NKG2D-CD123 T cells also showed significant cytotoxicity (88.28±6.58%) against K562-luc cells, while NKG2D T cells showed lower cytotoxicity (66.68±2.87%) and CD123 binder T cells had no cytotoxicity (−46.98±11.22%) against K562-Luc cells. These results indicate that NKG2D-CD123 T cells are more efficacious than NKG2D T cells.

Furthermore, cytotoxicity assays were carried out in the presence or absence of soluble MICA, a cognate ligand of NKG2D. NKG2D-CD123 T cells (LIC2002-2 or LIC2004) were blocked with recombinant MICA protein or BSA protein respectively when co-cultured with the target cells (K562-Luc or K562-CD123-Luc) at an E:T ratio of 20:1. MICA or BSA was added to the co-cultures at a concentration of 0 ng/mL, 100 ng/mL or 1000 ng/mL. T cells electroporated with no mRNA served as a negative control.

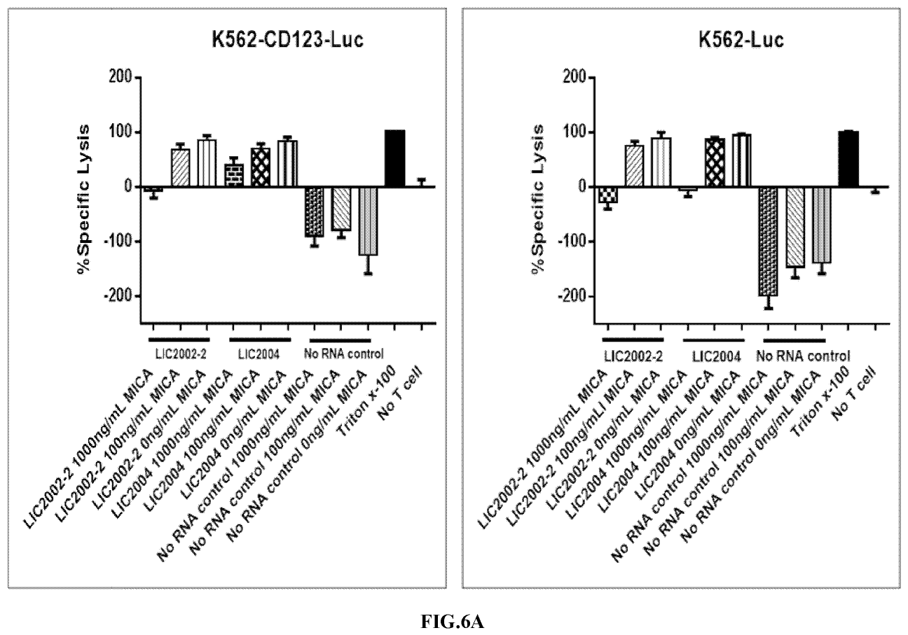

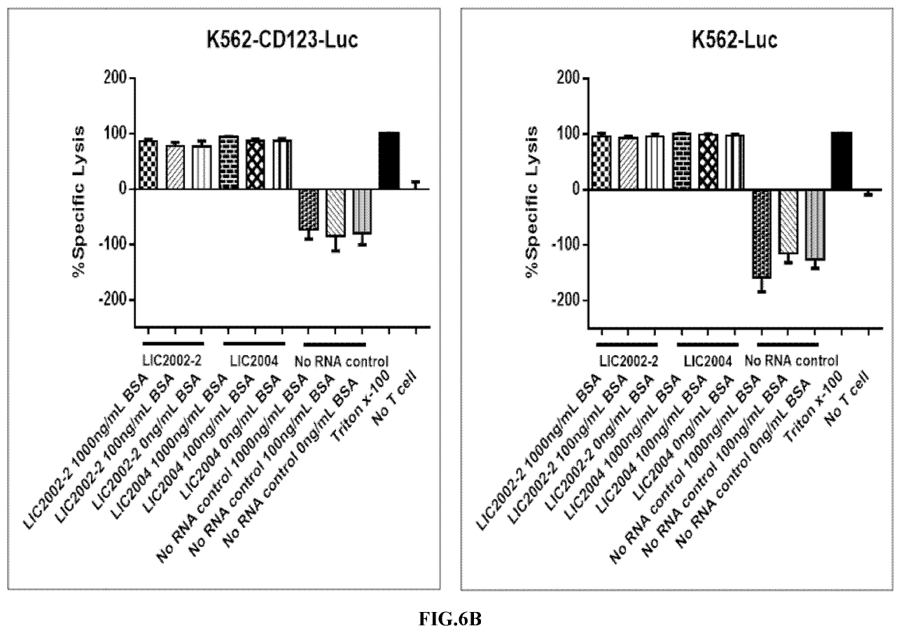

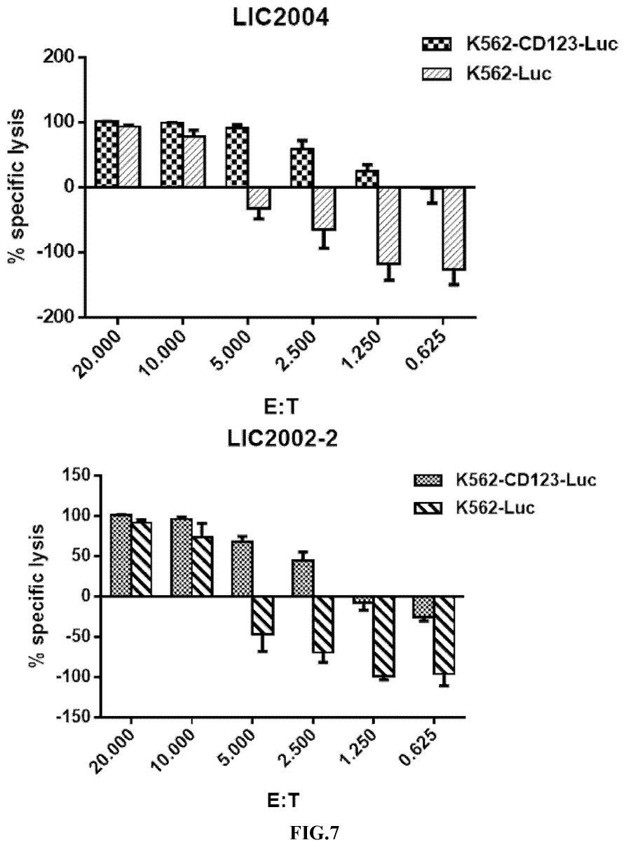

As shown in FIGS. 6A-6B, treatment with MICA at the highest concentration tested was able to block the NKG2D domain and reduce the cytotoxic activities of the LIC2002-2 and LIC2004 expressing T cells. Treatment with non-specific BSA did not significantly affect the cytotoxicity of the engineered T cells against tumor cells.

As shown in FIG. 7, LIC2002-2 expressing T cells and LIC2004 expressing T cells were co-incubated with K562-CD123-Luc or K562-Luc cell line at an effector (engineered T cells) to target cell ratio of 20:1, 10:1, 5:1, 2.5:1, 1.25:1 or 0.625:1. The results show dose-dependent killing effects of LIC2004 and LIC2002-2 on the K562 cell lines. Stronger killing effects were observed on K562 cell line expressing both NKG2D and CD123, than K562 cell line expressing NKG2D alone.

### IFNγ Release

Engineered T cells expressing the LIC2002-2, LIC2004 or LIC2004-1 constructs were co-incubated with K562-CD123-Luc, K562-Luc, or KG1-Luc cell lines for 20 h, respectively. Supernatants from the co-cultures were collected and assessed to determine levels of cytokine release (e.g., interferon gamma, IFNγ release).

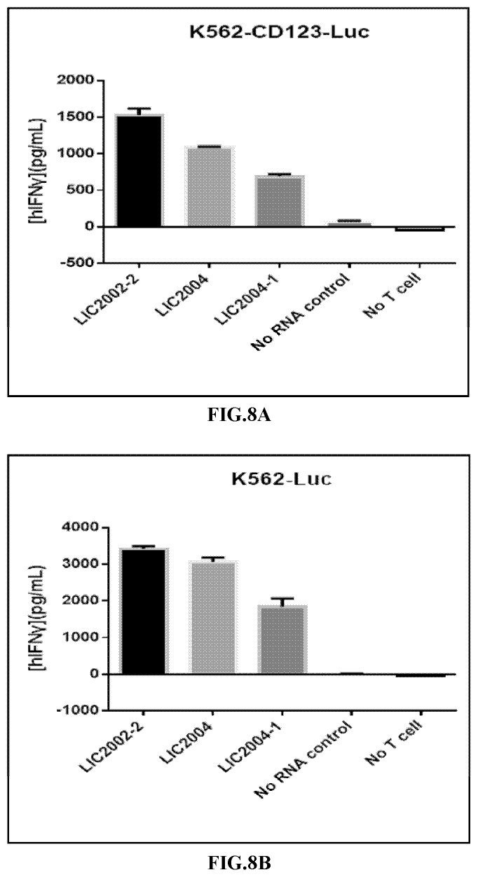

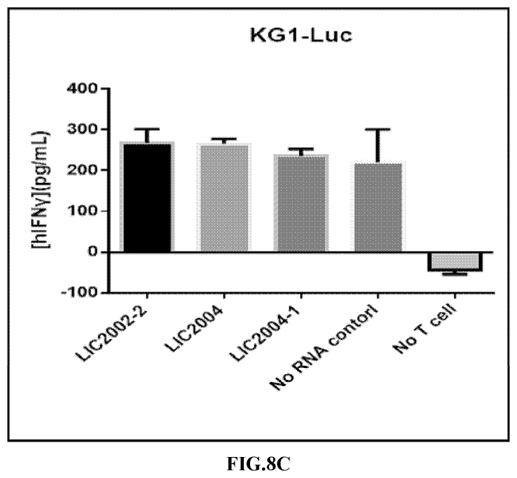

FIG. 8A shows the levels of IFNγ in cell-free supernatants after co-culture of the engineered T cells with CD123 positive K562-CD123-Luc for 20 hours. The secreted IFNγ levels were 1526.51±92.13 pg/mL (LIC2002-2), 1089.36±8.06 pg/mL (LIC2004), 687.62±31.65 pg/mL (LIC2004-1), and 64.15±16.56 pg/mL (no RNA control). IFNγ was not detectable in no T cell control.

FIG. 8B shows the levels of IFNγ in cell-free supernatants after co-culture of the engineered T cells with K562-Luc for 20 hours. The secreted IFNγ levels were 3416.67±71.15 pg/mL (LIC2002-2), 3063.46±119.46 pg/mL (LIC2004), 1841.41±222.18 pg/mL (LIC2004-1), and 3.99±11.57 pg/mL (no RNA control). IFNγ was not detectable in no T cell control.

FIG. 8C shows the levels of IFNγ in cell-free supernatants after co-culture of the engineered T cells with KG1-Luc for 20 hours. The secreted IFNγ levels were 267.75±34.33 pg/mL (LIC2002-2), 265.87±12.19 pg/mL (LIC2004), 236.56±16.45 pg/mL (LIC2004-1), and 220.56±80.5 pg/mL (no RNA control). IFNγ was not detectable in no T cell control.

What is claimed is:

1. A chimeric receptor comprising a polypeptide chain comprising:
(a) an extracellular domain comprising an NKG2D domain;
(b) a transmembrane domain; and
(c) an intracellular signaling domain.

2. A multispecific chimeric receptor comprising a polypeptide chain comprising:
(a) an extracellular domain comprising a first NKG2D domain, a second NKG2D domain, and a second antigen binding domain;
(b) a transmembrane domain; and
(c) an intracellular signaling domain.

3. The multispecific chimeric receptor of claim 2, wherein the extracellular domain comprises from the N-terminus to the C-terminus: the second antigen binding domain, the first NKG2D domain and the second NKG2D domain.

4. The chimeric receptor of any one of claims 1-3, wherein the second antigen binding domain is fused to the first NKG2D domain via a peptide linker.

5. A multispecific chimeric receptor comprising a first polypeptide chain and a second polypeptide chain each comprising:
(a) an extracellular domain comprising an NKG2D domain and a second antigen binding domain;
(b) a transmembrane domain; and
(c) an intracellular signaling domain.

6. The multispecific chimeric receptor of claim 5, wherein each extracellular domain further comprises a dimerization motif.

7. The multispecific chimeric receptor of claim 6, wherein the dimerization motif is displaced between the NKG2D domain and the second antigen binding domain.

8. The multispecific chimeric receptor of claim 6 or 7, wherein the dimerization motif is a leucine zipper.

9. The multispecific chimeric receptor of claim 8, wherein the second antigen binding domain is fused to the NKG2D domain via a peptide linker.

10. The multispecific chimeric receptor of any one of claims 5-9, wherein each of the first polypeptide chain and the second polypeptide chain comprises from the N-terminus to the C terminus: the second antigen binding domain, the NKG2D domain, the transmembrane domain, and the intracellular signaling domain.

11. A dual chimeric receptor system comprising:
(i) a first chimeric receptor comprising:
(a) a first extracellular domain comprising an NKG2D domain;
(b) a first transmembrane domain; and
(c) a first intracellular signaling domain; and
(ii) a second chimeric receptor comprising:
(a) a second extracellular domain comprising a second antigen binding domain; and
(b) a second transmembrane domain.

12. The dual chimeric receptor system of claim 11, wherein the second chimeric receptor further comprises a second intracellular signaling domain.

13. The multispecific chimeric receptor of any one of claims 2-10, or the dual chimeric receptor system of claim 11 or 12, wherein the second antigen binding domain is an antibody fragment.

14. The multispecific chimeric receptor or the dual chimeric receptor system of claim 13, wherein the antibody fragment specifically binds to an antigen selected from the group consisting of CD19, CD20, CD22, CD33, CD38, BCMA, CS1, ROR1, GPC3, CD123, CD138, c-Met, EGFR, EGFRvIII, HER2, HERS, GD-2, NY-ESO-1, MAGE A3, and glycolipid F77.

15. The multispecific chimeric receptor of any one of claims 2-10, or the dual chimeric receptor system of claim 11 or 12, wherein the second antigen binding domain is a ligand or a ligand binding domain.

16. The multispecific chimeric receptor or the dual chimeric receptor system of claim 15, wherein the ligand or ligand binding domain is derived from a molecule selected from the group consisting of NKG2A, NKG2C, NKG2F, IL-3, IL-13, LLT1, AICL, DNAM-1, and NKp80.

17. The multispecific chimeric receptor or the dual chimeric receptor system of claim 16, wherein the second antigen binding domain is an IL-3 domain.

18. The chimeric receptor of any one of claims 1-10 and 13-17, or the dual chimeric receptor system of any one of claims 11-17, wherein the NKG2D domain (or the first NKG2D domain and/or the second NKG2D domain) comprises the amino acid sequence of SEQ ID NO: 7 or 8.

19. The chimeric receptor of any one of claims 1-10 and 13-18, or the dual chimeric receptor system of any one of claims 11-18, wherein the transmembrane domain (or the first transmembrane domain and/or the second transmembrane domain) is derived from a molecule selected from the group consisting of CD8α, CD4, CD28, 4-1BB, CD80, CD86, CD152 and PD1.

20. The chimeric receptor of any one of claims 1-10 and 13-19, or the dual chimeric receptor system of any one of claims 11-19, wherein the intracellular signaling domain (or the first intracellular signaling domain and/or the second intracellular signaling domain) comprises a primary intracellular signaling domain of an immune effector cell.

21. The chimeric receptor of any one of claims 1-10 and 13-20, or the dual chimeric receptor system of any one of claims 11-20, wherein the intracellular signaling domain (or the first intracellular signaling domain and/or the second intracellular signaling domain) comprises a co-stimulatory signaling domain.

22. The chimeric receptor or the dual chimeric receptor system of claim 21, wherein the co-stimulatory signaling domain is derived from a co-stimulatory molecule selected from the group consisting of CD27, CD28, 4-1BB, OX40, CD30, ICOS, CD40, CD3, LFA-1, CD2, CD7, LIGHT, NKG2C, B7-H3, Ligands of CD83 and combinations thereof.

23. A chimeric receptor comprising an amino acid sequence having at least about 85% sequence identity to an amino acid sequence selected from the group consisting of SEQ ID NOs: 16-20 and 33-35.

24. A polypeptide comprising an amino acid sequence having at least about 85% sequence identity to the amino acid sequence of SEQ ID NO: 36 or 37.

25. An isolated nucleic acid comprising a nucleic acid sequence encoding the chimeric receptor of any one of claims 1-10 and 13-23.

26. An isolated nucleic acid encoding the dual chimeric receptor system of any one of claims 11-22, comprising a first nucleic acid sequence encoding the first chimeric receptor and a second nucleic acid sequence encoding the second chimeric receptor, wherein the first nucleic acid sequence is operably linked to the second nucleic acid sequence via a third nucleic acid sequence encoding a self-cleaving peptide.

27. An isolated nucleic acid comprising a nucleic acid sequence having at least about 85% sequence identity to a nucleic acid sequence selected from the group consisting of SEQ ID NOs: 21-27 and SEQ ID NOs: 38-40.

28. An engineered immune effector cell, comprising the chimeric receptor or the dual chimeric receptor system of any one of claims 1-23, or the isolated nucleic acid of any one of claims 25-27.

29. A pharmaceutical composition, comprising the engineered immune effector cell of claim 28, and a pharmaceutically acceptable carrier.

30. A method of treating a cancer in an individual, comprising administering to the individual an effective amount of the pharmaceutical composition of claim 29.
